(19) United States
(12) Patent Application Publication
(10) Pub. No.: US 2023/0339862 A1
(43) Pub. Date: Oct. 26, 2023

(54) NOVEL DIBENZOACRIDINIUM DERIVATIVES, THEIR PROCESS OF PREPARATION AND THEIR USE FOR TREATING VIRAL INFECTIONS

(71) Applicants: INSTITU NATIONAL DE LA SANTE ET DE LA RECHRCHE MEDICAL (INSERM), Paris (FR); UNIVERSITÉ DE BORDEAUX, Bordeaux (FR); CENTRE NATIONAL DE LA RECHERCHE SCIENTIFIQUE, Paris (FR)

(72) Inventors: Samir AMRANE, Talence (FR); Marie-Aline ANDREOLA, Bordeaux (FR); Céline OLIVIER, Talence (FR); Yann FERRAND, Pessac (FR)

(21) Appl. No.: 18/044,829

(22) PCT Filed: Sep. 14, 2021
(86) PCT No.: PCT/EP2021/075191

§ 371 Date: Mar. 10, 2023

(30) Foreign Application Priority Data
Sep. 14, 2020 (EP) 20306022.3

(51) Int. Cl. C07D 221/18; C07D 401/06; A61P 31/18; A61P 31/22

(52) U.S. Cl. CPC C07D 221/18; C07D 401/06; A61P 31/18; A61P 31/22

(57) ABSTRACT

The present invention relates to novel candidates for treating viral infections such as HIV, Epstein Barr virus, HPV (Papillomavirus), SARS coronavirus, Ebola virus, Marburg virus, Zika, Herpes (HHV), Hepatitis B, Hepatitis C, Kaposi's sarcoma-associated herpesvirus (KSHV). In particular, the invention relates to novel dibenzoacridinium derivatives that are shown to be G-quadruplex ligands and are thus useful for treating the above infections. Also the invention relates to the process of preparation of the novel dibenzoacridinium derivatives.

The present invention concerns derivatives of acridinium, their preparation and their application in therapeutics.

More specifically, this application is related to the use of acridinium derivatives as G-quadruplex ligands to inhibit viral infections, such as HIV, more particularly to inhibit HIV-1 replication cycle or filoviruses.

Guanine rich RNA or DNA sequences are capable of folding and adopting four stranded structures called G-quadruplexes, or “G4”. These unusual nucleic acid structures are based on the stacking of 2, 3 or 4 tetrads; each of which is composed of four guanines connected by 8 hydrogen bonds. These tetrads are stabilized by the presence of a central cation, K⁺ or Na⁺, abundantly present in the cellular environment, and which is coordinated with the oxygens of the carbonyl groups. Numerous thermodynamic studies have shown that these structures are very stable. They often have thermal denaturation temperatures above 50° C., and some are stable at 90° C. (Guedin et al Nucleic Acids Res 2009, 37, 5559). Once formed, some G4 such as the one formed by the c-myc promoter sequence, have a very long half-life and can withstand the annealing in the presence of high excess of their complementary strand (up to 50 times). Polymorphism, robustness and fast folding are some of the intrinsic characteristics of the G4 which strongly suggest a biological role. Several bioinformatics studies have determined the distribution of quadruplex motifs in the human genome (Bedrat et al Nucleic Acids Res 2016, 44, 1746): i) The human genome has 1 000 000 potential quadruplexes motifs, ii) 40% of the genes encoding a protein have at least one quadruplex located at 1 kb from initiation site of the transcription, iii) important transcription factor binding sites, such as SP1, MAZ, Krox ZF5 are positioned near or overlap the G4 motifs, iv) 37% of the preferential recombination sites present a G4 motif. These correlations are conserved among different species in the evolution of the genomes.

Recently several in vivo studies have confirmed the existence of G4 in the genomic DNA (Biffi et al S. Nat Chem 2013, 5, 182. and cellular RNA (Biffi et al Nat Chem 2014, 6, 75). Some studies have used fluorescent probes (antibodies or ligands) which specifically recognize G4s and do not bind to other DNA structures. These probes allowed the direct detection of G4s structures in immobilized chromosomes and confirmed the presence of quadruplexes in the regulatory regions of genes and subtelomeric regions. The involvement of G4s in replication, transcription, RNA splicing, or translation has also been extensively studied.

Quadruplexes have been identified as therapeutic targets (Neidle et al. J Med Chem 2016, 59, 5987), as follows:

a) Inhibition of Telomerase

Targeting G-quadruplexes was initiated in order to inhibit telomerase, a enzyme reactivated in 85% of cancers, but inactive in most normal cells. This enzyme recognizes the human telomeric sequence in its single-stranded conformation and maintains telomere length in tumor cells which makes them “immortal”. The strategy is to stabilize telomeric G4s with chemicals to prevent telomerase interaction with its substrate. G4s ligands with anticancer properties have already been discovered, it is the case of Braco19, RHPS4 20 and 360A.

b) Inhibitions of Oncogenes

Many laboratories have been interested in quadruplex targeting to inhibit the expression of oncogenes. The oncogene c-myc is an important target of this approach: it has a G4 sequence in its promoter and the expression level of the gene depends on the formation of this structure. The formation of the G4 represses the transcription and this inhibition is enhanced in the presence of G4 ligands. The same type of repressive effect of the ligand was observed for KRAS, c-kit and bcl2 oncogenes.

c) Targeting G4s

The unique features of the G4 topology, very distinct from a DNA or RNA duplex or single-strand, make it a therapeutic target. G4s are compact structures which targeting can be likened to that of globular proteins. The great structural diversity of G4 suggests that a relatively high degree of selectivity can be achieved. Examples of rational design of ligands and in silico screening are becoming more numerous in the literature. This strategy opens a promising new era of targeting offering an alternative to the usual proteins targeting strategy. Furthermore, if the first applications were related only to cancer, new applications of this research are now considered in virology.

In a recent review, Harris et al proposed that the G4s may have a biological role in the life cycle of different pathogens (Harris & Merrick, PLoS Pathog. 2015 11(2):e1004562. The inventors also wrote a review describing the role of G4s in the replication cycle of many viruses (Métifiot et al Nucleic Acid Res. 2014 42, 12352-66). In the case of SARS coronavirus, a viral protein called “single domain SARS” (SUD) has two G4 binding sites. This viral protein seems essential for the virulence of the virus, would fight the immune response of the host by targeting the quadruplexes of the latter. In the case of Epstein-Barr virus EBNA1 viral protein binds to the G4 RNA and is involved in the viral replication cycle. Finally, HPV also presents G4 sequences in its genome.

The RNA genome of HIV-1 is changing very quickly, which gives it an important structural variability allowing it to escape the immune response of the infected organism. The low fidelity of the reverse transcriptase and the many genetic recombination events between the two viral RNAs are the drivers of this trend. These recombinations are facilitated by the dimerization of the viral RNA at the DIS sequence (Dimer Initiation Site). A recent study has suggested that this recombination could also be done via a bimolecular quadruplex using the cPPT sequences of each of the viral RNAs. In another study, a preferential recombination site was found at the 5′ region of the gene gag. This guanine-rich sequence is capable of forming a bimolecular quadruplex with the homologous sequence on the other strand. This quadruplex facilitates the exchange of material between the donor RNA and the recipient RNA. NCp7 protein is known to facilitate the packaging of the viral RNA, the reverse transcription and integration into the genome, but is also able to open intramolecular RNA structures to promote the bimolecular structures. The NCp7 can also promote the formation of a bimolecular G4 with a receiver RNA.

In a previous application (EP14305763.6), the inventors showed that, despite its high genetic variability, HIV-1 genome presents several very conserved G4 forming sequences. These G4 sequences are associated with critical regulatory functions of the HIV replication cycle such as i) the initiation of reverse transcription by forming the central initiation point of the (+) strand synthesis by the reverse transcriptase; ii) the initiation of reverse transcription by forming the first point of (+) strand synthesis by the reverse transcriptase; iii) the regulation of the transcription of the provirus. The HIV virus was confronted to synthetic G4 DNA and RNA derived from its own genome. The observed inhibitory effects suggest that these synthetic “viral” G4s act as decoys diverting viral or cellular proteins from their natural targets in the viral genome.

The initial applications of G4 ligands were mostly related to cancer. However, in 2014, new applications in virology were suggested by the inventors (Amrane et al J Am Chem Soc 2014, 136, 5249). Since then, an important number of publications described the antiviral effects of several ligands suggesting potential new therapeutic avenues. Notably, as recently reviewed in Nucleic Acids Research (Ruggiero et al Nucleic Acids Res 2018), G4 ligands of various chemical families (Bisquinoliniums, naphthalene diimides, acridiniums) were able to inhibit DNA viruses such as herpesviruses (HSV, EBV) and Hepatitis B virus as well as RNA viruses such as HIV-1 and Hepatitis C virus. These effects suggest that G4s structures are likely formed in the viral genome. However, direct evidences are still missing regarding the G4 based mechanism of action.

Ruggiero et al (Nucleic Acids Research, 2018, 1-14) disclose G4 ligands such as Braco-19.

It is desirable to provide novel G4-ligands to achieve candidates useful for treating viral infections.

According to a first object, the present invention concerns a compound of formula (I):

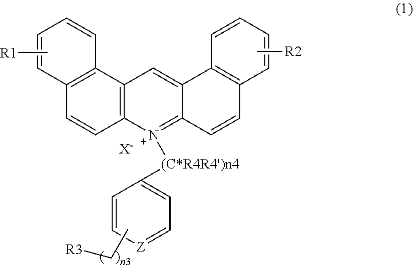

wherein
R1, R2 and R3 are identical or different and may be located on any position of the benzene ring on which they are attached;
R1, R2 independently represent H, —O(C1-C6)Alkyl, OH, (C1-C6)alkyl, COOR, NO₂, CN, NRR′;
R3 is chosen from H, COO(C1-C6)Alkyl, —O(C1-C6)Alkyl, NO₂, (C1-C6)alkyl, OH, COOH, CN, NRR′, CF₃, NRR′R″⁺/Y⁻, or a guanidine group chosen from

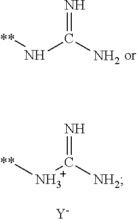

** illustrates the attachment of the guanidine group to the terminal carbon of the —(CH₂)ₙ₃-chain;
C* denotes an optionally asymmetric carbon atom;
R4 and R4′, identical or different independently represent H, (C1-C6)alkyl;
n3 and n4, identical or different are independently chosen from integer comprised between 0 and 6;
Z represents CH or N, or C or N⁺/X′⁻ when R3 is not H;
R, R′ and R″, identical or different independently represent H, (C1-C6)alkyl;
X⁻, X′⁻ and Y⁻ independently represent an anion;

Optionally in the form of a hydrate or of a solvate thereof.

In particular, it concerns a compound of formula (I):

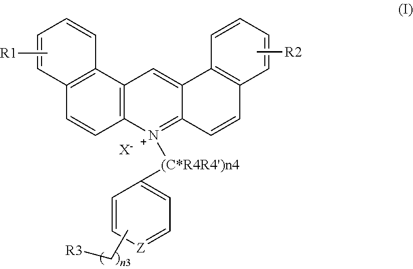

wherein
R1, R2 and R3 are identical or different and may be located on any position of the benzene ring on which they are attached;
R1, R2 independently represent H, —O(C1-C6)Alkyl, OH, (C1-C6)alkyl, COOR, NO₂, CN, NRR′;
R3 is chosen from H, COO(C1-C6)Alkyl, —O(C1-C6)Alkyl, NO₂, (C1-C6)alkyl, OH, COOH, CN, NRR′, CF₃, NRR′R″⁺/Y⁻; or a guanidine group chosen from

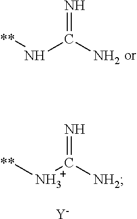

** illustrates the attachment of the guanidine group to the terminal carbon of the —(CH₂)ₙ₃-chain;
C* denotes an optionally asymmetric carbon atom;
R4 and R4′, identical or different independently represent H, (C1-C6)alkyl;
n3 and n4, identical or different are independently chosen from integer comprised between 0 and 6;
R, R′ and R″, identical or different independently represent H, (C1-C6)alkyl;
X⁻ and Y⁻ independently represent an anion;

Optionally in the form of a hydrate or of a solvate thereof.

According to an embodiment, the compound of formula (I) is of formula (IA):

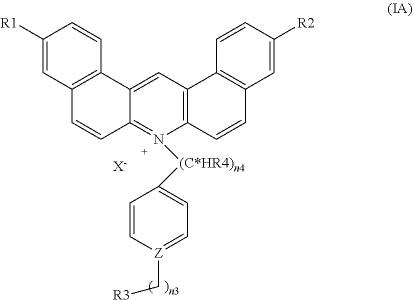

wherein
R1, R2, R3, R4, X, Z, n3 are defined as in claim 1; and n4 is 0 or 1.

In particular, it is of formula (IA):

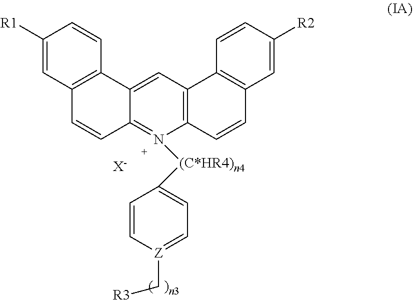

wherein
R1, R2, R3, R4, X⁻, n3 are defined as in formula (I) and n4 is 0 or 1.

According to an embodiment, in formula (I) or (IA):
R1. R2 independently represent H. —O(C1-C6)Alkyl;
R3 is chosen from H, COO(C1-C6)Alkyl, —O(C1-C6)Alkyl, NO₂;
R4 represents H or (C1-C6)Alkyl and R4′ represents H;
n3 is 0;
n4 is 0 or 1;
R, R′ and R″, identical or different, independently represent H, (C1-C6)alkyl;
X represents a halogen atom; and
Z represents CH or N, or C or N⁺/X′⁻ when R3 is not H;
or an alternative pharmaceutically acceptable salt thereof.

According to a particular embodiment, the compound of formula (I) is chosen from the following group of compounds:

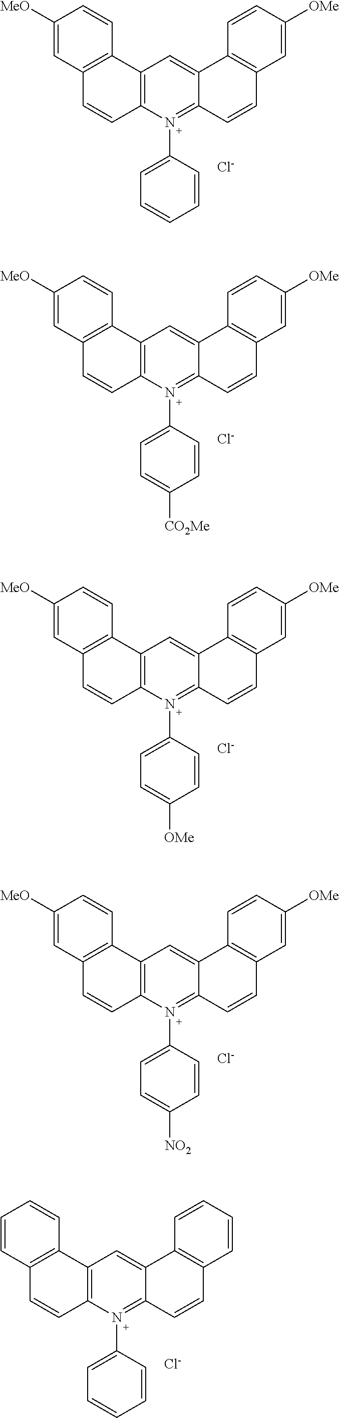

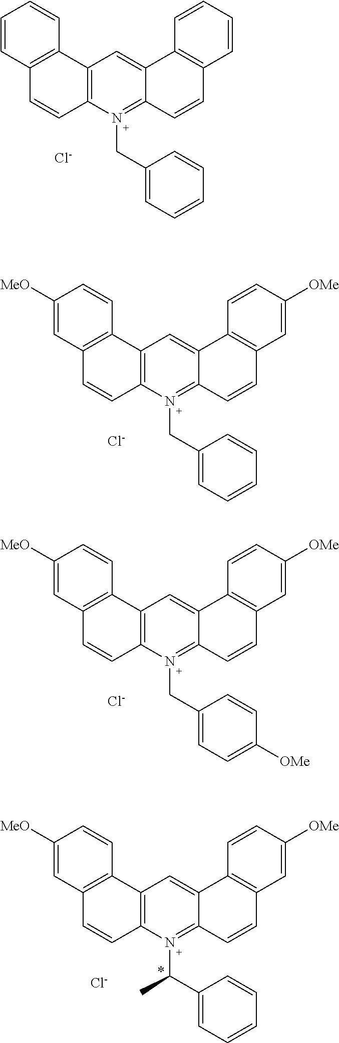

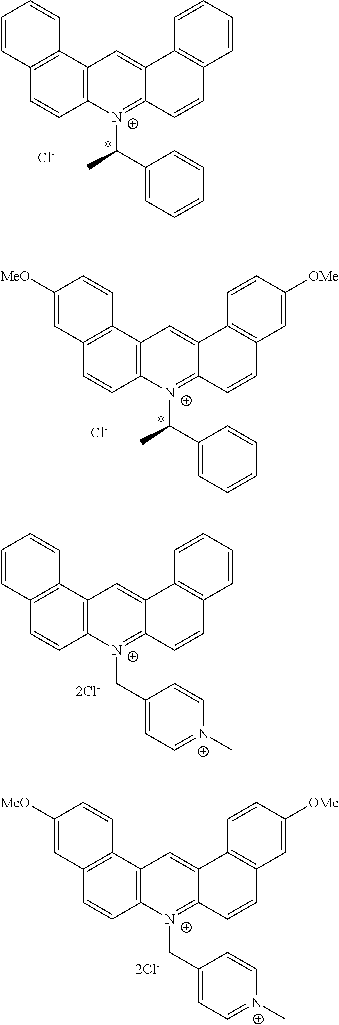

or alternative pharmaceutically acceptable salts thereof.

Unless specified otherwise, the terms used hereabove or hereafter have the meaning ascribed to them below:

The anion refers herein may be chosen with halides or any other anion such as PO₂Cl₂⁻, PF₆⁻, BF₄⁻, [BArF₄]⁻ etc

“Halo”, “hal” or “halogen” refers to fluorine, chlorine, bromine or iodine atom.

“Halide” refers to the anion of a halogen atom.

“Alkyl” represents an aliphatic-hydrocarbon group which may be straight or branched having 1 to 6 carbon atoms in the chain. In a particularly preferred embodiment the alkyl group has 1 to 4 carbon atoms in the chain. Exemplary alkyl groups include methyl, ethyl, n-propyl, iso-propyl, iso-butyl, n-butyl, tert-butyl, n-pentyl, 3-pentyl.

The compounds of formula (I) can be provided in the form of addition salts with acids, which also form part of the invention.

The compounds of the present invention may possess an acidic group and a basic group which may form corresponding salts. Thus the present invention includes salts of compounds of formula (I). The salts may preferably be pharmaceutically acceptable salts. The acidic group may form salts with bases. The base may be an organic amine base, for example triethylamine, tert-butylamine, tromethamine, meglumine, epolamine, etc. The acidic group may also form salts with inorganic bases like sodium hydroxide, potassium hydroxide, etc. The basic group may form salts with inorganic acids like hydrochloric acid, sulfuric acid, hydrobromic acid, sulfamic acid, phosphoric acid, nitric acid etc and organic acids like acetic acid, propionic acid, succinic acid, tartaric acid, citric acid, methanesulfonic acid, benzenesulfonic acid, glucoronic acid, glutamic acid, benzoic acid, salicylic acid, toluenesulfonic acid, oxalic acid, fumaric acid, maleic acid etc. Further, compounds of formula (I) may form quaternary ammonium salts and salts with amino acids such as arginine, lysine, etc. Lists of suitable salts may be found in Remington's Pharmaceutical Sciences, 17th ed., Mack Publishing Company, Easton, P A, 1985, p. 1418 and P. H. Stahl, C. G. Wermuth, Handbook of Pharmaceutical salts—Properties, Selection and Use, Wiley-VCH, 2002, the disclosures of which are hereby incorporated by reference.

These salts are advantageously prepared with pharmaceutically acceptable acids, but salts with other acids, useful for example for the purification or for the isolation of the compounds of formula (I), also form part of the invention.

The compounds of formula (I) can comprise one or more asymmetric carbon atoms herein denoted C*. They can therefore exist in the form of enantiomers or diastereoisomers. These enantiomers and diastereoisomers, as well as their mixtures, including racemic mixtures, form part of the invention.

It is well known in the art how to prepare and isolate such optically active forms. For example, mixtures of stereoisomers may be separated by standard techniques including, but not limited to, resolution of racemic forms, normal, reverse-phase, and chiral chromatography, preferential salt formation, recrystallization, and the like, or by chiral synthesis either from chiral starting materials or by deliberate synthesis of target chiral centers.

The compounds of formula (I) can also be provided in the form of a hydrate or of a solvate, i.e. in the form of associations or combinations with one or more water or solvent molecules. Such hydrates and solvates also form part of the invention.

According to another object, the present invention concerns the process of preparation of a compound of formula (I) according to the invention as defined above.

The compounds and process of the present invention may be prepared in a number of ways well known to those skilled in the art. The compounds can be synthesized, for example, by application or adaptation of the methods described below, or variations thereon as appreciated by the skilled artisan. The appropriate modifications and substitutions will be readily apparent and well known or readily obtainable from the scientific literature to those skilled in the art. In particular, such methods can be found in R. C. Larock, Comprehensive Organic Transformations, VCH publishers, 1989

The reagents and starting materials may be commercially available, or readily synthesized by well-known techniques by one of ordinary skill in the arts. All substituents, unless otherwise indicated, are as previously defined.

In the reactions described hereinafter, it may be necessary to protect reactive functional groups, for example hydroxy, amino, imino, thio or carboxy groups, where these are desired in the final product, to avoid their unwanted participation in the reactions. Conventional protecting groups herein named Pg may be used in accordance with standard practice, for examples see T. W. Greene and P. G. M. Wuts in Protective Groups in Organic Synthesis, John Wiley and Sons, 1991; J. F. W. McOmie in Protective Groups in Organic Chemistry, Plenum Press, 1973.

Some reactions may be carried out in the presence of a base. There is no particular restriction on the nature of the base to be used in this reaction, and any base conventionally used in reactions of this type may equally be used here, provided that it has no adverse effect on other parts of the molecule. Examples of suitable bases include: sodium hydroxide, potassium carbonate, triethylamine, alkali metal hydrides, such as sodium hydride and potassium hydride; alkyllithium compounds, such as methyllithium and butyllithium; and alkali metal alkoxides, such as sodium methoxide and sodium ethoxide.

Usually, reactions are carried out in a suitable solvent. A variety of solvents may be used, provided that it has no adverse effect on the reaction or on the reagents involved. Examples of suitable solvents include: hydrocarbons, which may be aromatic, aliphatic or cycloaliphatic hydrocarbons, such as hexane, cyclohexane, benzene, toluene and xylene; amides, such as dimethyl-formamide; alcohols such as ethanol and methanol and ethers, such as diethyl ether and tetrahydrofuran.

The reactions can take place over a wide range of temperatures. In general, it was found convenient to carry out the reaction at a temperature of from 0° C. to 150° C. (more preferably from about room temperature to 100° C.). The time required for the reaction may also vary widely, depending on many factors, notably the reaction temperature and the nature of the reagents. However, provided that the reaction is effected under the preferred conditions outlined above, a period of from 3 hours to 20 hours will usually suffice.

The compound thus prepared may be recovered from the reaction mixture by conventional means. For example, the compounds may be recovered by distilling off the solvent from the reaction mixture or, if necessary after distilling off the solvent from the reaction mixture, pouring the residue into water followed by extraction with a water-immiscible organic solvent and distilling off the solvent from the extract. Additionally, the product can, if desired, be further purified by various well-known techniques, such as recrystallization, reprecipitation or the various chromatography techniques, notably column chromatography or preparative thin layer chromatography.

In particular, the process of preparation of a compound of formula (I) comprises the step of reacting a compound of formula (II)

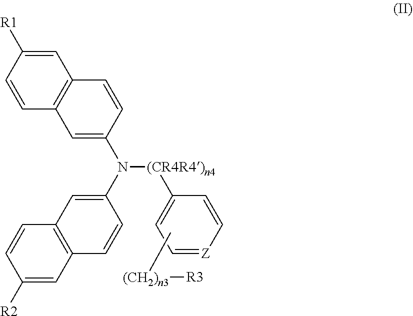

with a halogenating agent and DMF
Where X, R1, R2, R3, R4, R4′, Z, n3 and n4 are defined as in formula (I)
and optionally isolating the compound of formula (I) that has been formed.

According to an embodiment, the halogenating agent may be of formula POX₃, such as POCl₃ or POBr₃ which is commercially available (Sigma-Aldrich, ACROS, ALFA AESAR, TCI etc. . . . ). Compounds with alternative X may be obtained from corresponding compounds with X═Br or Cl, by anion exchange (metathesis).

According to an embodiment, the compound of formula (II) where n is 0 may be prepared by conducting a Buchwald-Hartwig coupling reaction, by application or adaptation of the procedure described by Olivier et al., ChemSusChem 2011, 4, 731.

Typically, the compound of formula (II) where n is 0 may be obtained by reacting a compound of formula (IV):

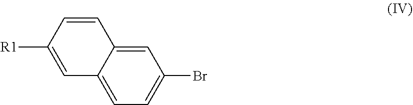

And a compound of formula (V):

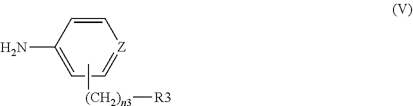

Optionally followed when R2 and R1 are different, by reacting the obtained compound with a compound of formula (IV′):

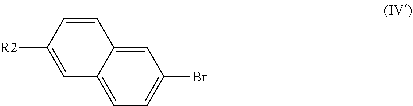

Where R1, R2, R3, n3 are defined as in formula (I),

Where each step is conducted in the presence of P(tBu)₃/Cs₂CO₃, and catalyzed by palladium such as with Pd(OAc)₂.

Typically, this reaction may be conducted in an organic solvent, such as toluene.

According to an embodiment, the step reacting the compound (IV) is conducted in equimolar conditions.

According to an alternative embodiment, the compound of formula (II) where n is not 0 may be prepared by reacting a compound of formula (III):

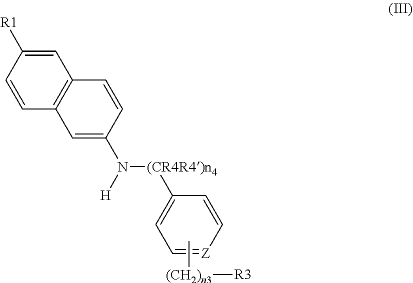

With a compound of formula (IV′)

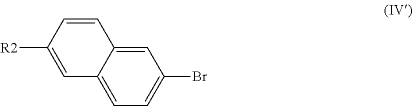

Where R1, R3, R4, R4′, n3 are defined as in formula (I) and n4 is an integer from 1 to 6, In the presence of P(tBu)₃/Cs₂CO₃ and catalyzed by palladium such as Pd(OAc)₂.

The compound of formula (III) may be in turn obtained by reacting the corresponding compounds of formula (IV) and (V′):

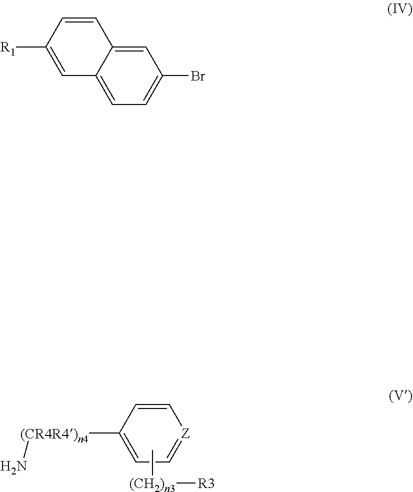

in a Ullmann coupling, catalyzed by copper. This reaction may be conducted by application or adaptation of the procedure reported by Ma et al., Org. Lett. 2003, 5, 2453, typically by reacting the starting compounds with CuI/K₂CO₃ and L-proline. Generally, this reaction may be carried out in an organic solvent, such as DMSO.

More particularly, the compounds of formula (II), (V), (III), (V′) are respectively of formula:

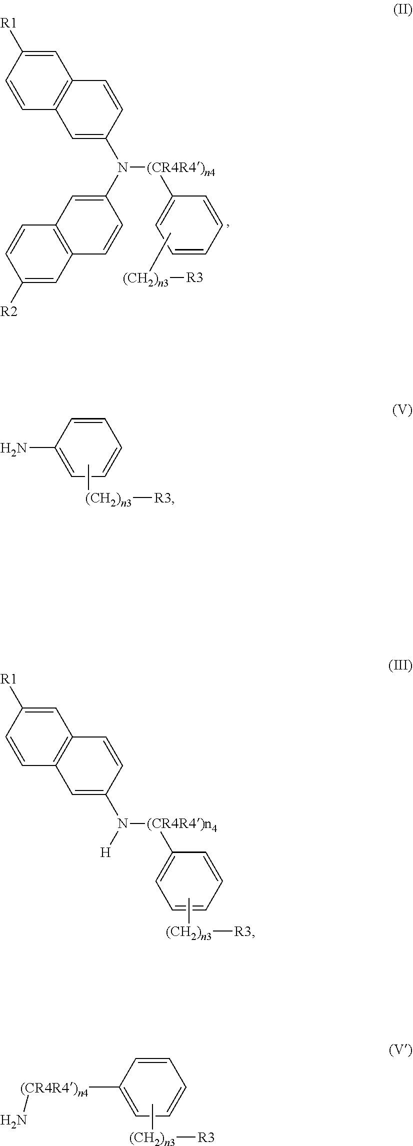

Generally, the products of formula (IV), (IV′), (V), (V′) and the reagents described above are commercially available.

According to another object, the present invention concerns a pharmaceutical composition comprising a compound of formula (I) according to the invention as defined above, together with at least one pharmaceutically acceptable excipient.

According to a further object, the present invention concerns a compound of formula (I) as defined above for its use in the prevention and/or treatment of a viral infection.

Viral infections include all disorders caused by a viruses which comprise G quadruplex sequences in their genome at the DNA or RNA levels. Viral infections include in particular HIV, Epstein Barr virus, HPV (Papillomavirus), SARS coronavirus, Ebola virus, Marburg virus, Zika, Herpes (HHV), Hepatitis B, Hepatitis C, Kaposi's sarcoma-associated herpesvirus (KSHV).

According to an embodiment, the present invention also concerns the use of a compound of formula (I) according to the invention for the preparation of a medicament for treating and/or preventing a viral infection.

According to a further embodiment, the present invention also concerns a method of treatment and/or prevention of a viral infection comprising the administration of a compound of formula (I) according to the invention as defined above to a patient in the need thereof.

As used herein, the term “patient” refers to a warm-blooded animal such as a mammal, preferably a human or a human child, which is afflicted with, or has the potential to be afflicted with one or more diseases and conditions described herein.

As used herein, a “therapeutically effective amount” refers to an amount of a compound of the present invention which is effective in reducing, eliminating, treating or controlling the symptoms of the herein-described diseases and conditions. The term “controlling” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment and chronic use.

As used herein, the expression “pharmaceutically acceptable” refers to those compounds, materials, compositions, or dosage forms which are, within the scope of sound medical judgment, suitable for contact with the tissues of human beings and animals without excessive toxicity, irritation, allergic response, or other problem complications commensurate with a reasonable benefit/risk ratio.

The dosage of drug to be administered depends on such variables as the type and extent of progression of the disease or disorder, the overall health status of the particular patient, the relative biological efficacy of the compound selected, and formulation of the compound, excipients, and its route of administration.

The compounds of present invention may be formulated into a pharmaceutically acceptable preparation, on admixing with a carrier, excipient or a diluent, in particular for oral or parenteral use. Oral preparations may be in the form of tablets, capsules or parenterals. A solid carrier can include one or more substances which may also act as flavoring agents, lubricants, solubilizers, suspending agents, fillers, glidants, compression aids, binders or tablet-disintegrating agents; it can also be an encapsulating material. Liquid carriers can include water, an organic solvent, a mixture of both or pharmaceutically acceptable oils and fats. The compositions may conveniently be administered in unit dosage form and may be prepared by any of the methods well known in the pharmaceutical art, for example, as described in Remington: The Science and Practice of Pharmacy, 20th ed.; Gennaro, A. R., Ed.; Lippincott Williams & Wilkins: Philadelphia, P A, 2000. Pharmaceutically compatible binding agents and/or adjuvant materials can be included as part of the composition.

The tablets, pills, powders, capsules, troches and the like can contain one or more of any of the following ingredients, or compounds of a similar nature: a binder such as microcrystalline cellulose, or gum tragacanth; a diluent such as starch or lactose; a disintegrant such as starch and cellulose derivatives; a lubricant such as magnesium stearate; a glidant such as colloidal silicon dioxide; a sweetening agent such as sucrose or saccharin; or a flavoring agent such as peppermint, or methyl salicylate. Preferred tablets contain lactose, cornstarch, magnesium silicate, croscarmellose sodium, povidone, magnesium stearate, or talc in any combination. Capsules can be in the form of a hard capsule or soft capsule, which are generally made from gelatin blends optionally blended with plasticizers, as well as a starch capsule. In addition, dosage unit forms can contain various other materials that modify the physical form of the dosage unit, for example, coatings of sugar, shellac, or enteric agents. Other oral dosage forms syrup or elixir may contain sweetening agents, preservatives, dyes, colorings, and flavorings. In addition, the active compounds may be incorporated into fast dissolve, modified-release or sustained-release preparations and formulations, and wherein such sustained-release formulations are preferably bi-modal.

Liquid preparations for administration include sterile aqueous or non-aqueous solutions, suspensions, and emulsions. The liquid compositions may also include binders, buffers, preservatives, chelating agents, sweetening, flavoring and coloring agents, and the like. Non-aqueous solvents include alcohols, propylene glycol, polyethylene glycol, acrylate copolymers, vegetable oils such as olive oil, and organic esters such as ethyl oleate. Aqueous carriers include mixtures of alcohols and water, hydrogels, buffered media, and saline. In particular, biocompatible, biodegradable lactide polymer, lactide/glycolide copolymer, or polyoxyethylene-polyoxypropylene copolymers may be useful excipients to control the release of the active compounds. Intravenous vehicles can include fluid and nutrient replenishers, electrolyte replenishers, such as those based on Ringer's dextrose, and the like. Other potentially useful parenteral delivery systems for these active compounds include ethylene-vinyl acetate copolymer particles, osmotic pumps, implantable infusion systems, and liposomes.

Other features of the invention will become apparent in the course of the following description of exemplary embodiments that are given for illustration of the invention and not intended to be limiting thereof.

### DESCRIPTION OF THE FIGURES

FIG. 1 illustrates the principle of the stabilization test by FRET to evaluate the binding of the ligands: The transfer of fluorescence energy between fluorescein and tetramethylrhodamine is possible when two fluorophores are close in the folded state.

FIG. 2 represents the radar plot representation of the stabilisation (ΔTm ° C.) induced by the ligand for the G4-forming oligonucleotide and the hairpin forming oligonucleotide.

FIG. 3 shows an example of HIV-1 inhibition curve induced by the compounds of the invention, and comparative compound BRACO 19. The cells are incubated with the ligand 30 minutes and then infected with HIV-1. After 24 hours the activity of beta-galactosidase, which is proportional to the infectivity of the virus is measured on a fluorescence plate reader.

FIG. 4 analyses the cytotoxicity effect induced by the ligands on the HeLaP4 cells in the absence of virus.

FIG. 5 demonstrates the inhibition of EBOV and MBGV by acridinium derivatives of the invention. A) EBOV and B) MGBV inhibition measured by RT-qPCR. Some of these experiments have been conducted 2 times using 2 different stocks of viruses at 5 months interval. C) EBOV and MGBV inhibition measured by immunohistochemistry for compound CO39.

The above mentioned features of the invention are given for illustration of the invention and not intended to be limiting thereof.

The following examples describe the synthesis of some compounds according to the invention. These examples are not intended to be limitative and only illustrate the present invention.

### Examples

I—Chemical Synthesis

The following compounds of formula (I) were synthesized
co-31: R₁═R₂═OMe, R₃═H; n3=n4=0;
co-330: R1═R2═OMe, R3═CO2Me; n3=n4=0;
co-360: R1═R2═R3═OMe; n3=n4=0;
co-370: R1═R2═OMe, R3═NO₂; n3=n4=0;
co-38: R1═R2=R3═H; n3=n4=0;
co-39: R1═R2=R3═H, R4═R4′═H; n3=0, n4=1;
co-397: R1═R2═OMe, R3═H, R4═R4′═H; n3=0, n4=1;
co-398: R1═R2═R3═OMe, R4═R4′═H; n3=0, n4=1;
co-399: R1═R2═OMe, R3═H, R4═H, R4′═CH3; n3=0, n4=1;
according to the following general procedure:

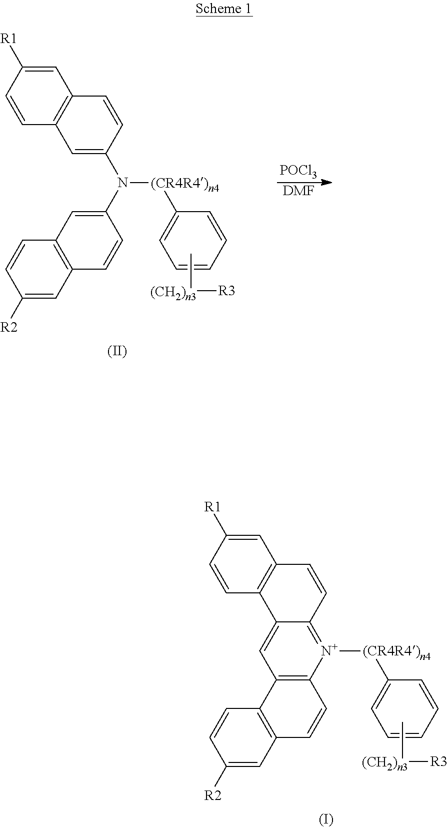

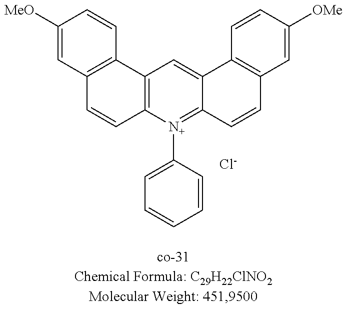 [SMILES: COc1ccc2c(ccc3c2cc2c4ccc(OC)cc4ccc2[n+]3-c2ccccc2)c1 ; [Cl-]]

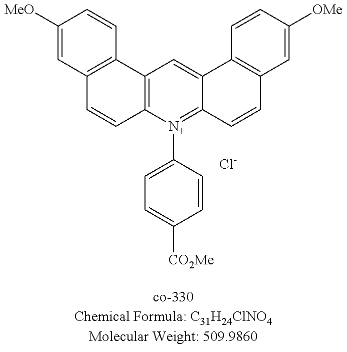 [SMILES: COC(=O)c1ccc(-[n+]2c3ccc4cc(OC)ccc4c3cc3c4ccc(OC)cc4ccc32)cc1 ; [Cl-]]

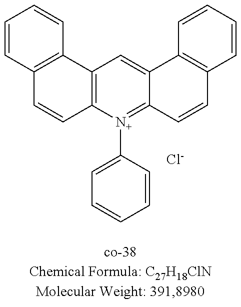 [SMILES: c1ccc(-[n+]2c3ccc4ccccc4c3cc3c4ccccc4ccc32)cc1 ; [Cl-]]

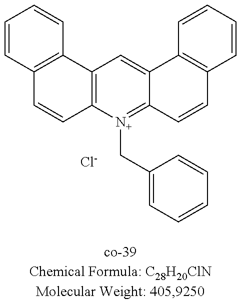 [SMILES: c1ccc(C[n+]2c3ccc4ccccc4c3cc3c4ccccc4ccc32)cc1 ; [Cl-]]

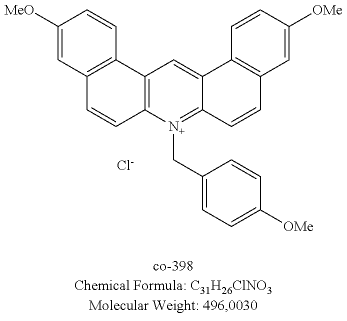 [SMILES: COc1ccc(C[n+]2c3ccc4cc(OC)ccc4c3cc3c4ccc(OC)cc4ccc32)cc1 ; [Cl-]]

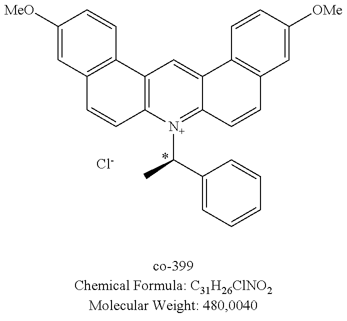 [SMILES: COc1ccc2c(ccc3c2cc2c4ccc(OC)cc4ccc2[n+]3[C@H](C)c2ccccc2)c1 ; [Cl-]]

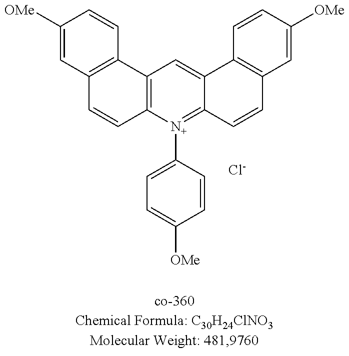 [SMILES: COc1ccc(-[n+]2c3ccc4cc(OC)ccc4c3cc3c4ccc(OC)cc4ccc32)cc1 ; [Cl-]]

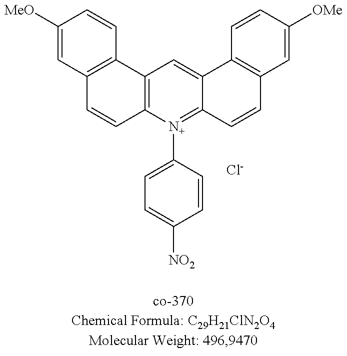 [SMILES: COc1ccc2c(ccc3c2cc2c4ccc(OC)cc4ccc2[n+]3-c2ccc([N+](=O)[O-])cc2)c1 ; [Cl-]]

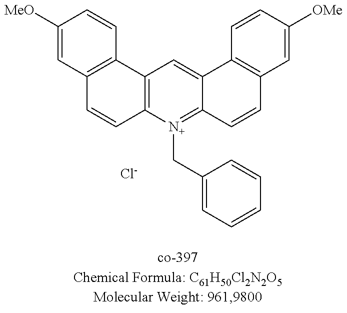 [SMILES: COc1ccc2c(ccc3c2cc2c4ccc(OC)cc4ccc2[n+]3Cc2ccccc2)c1 ; [Cl-]]

I.1 Materials and Methods

All reagents and starting materials of formula (IV), (IV′), (V), (V′) were obtained from commercially available sources (SIGMA-ALDRICH, ALFA-AESAR, TCI CHEMICALS) and used without further purification. Solvents were dried from appropriate drying agents (sodium for toluene, calcium hydride for dichloromethane) and freshly distilled before use. Dimethylformamide was purified through azeotropic distillation with water and benzene.

¹H NMR and ¹³C NMR analyses were performed on Bruker Avance 300 and DPX 400 spectrometers. Chemical shift values are given in ppm with reference to solvent residual signals. HR-MS analyses were performed on a Qstar spectrometer at CESAMO (Bordeaux).

I.2 Synthetic Routes to the Dibenzoacridinium Derivatives

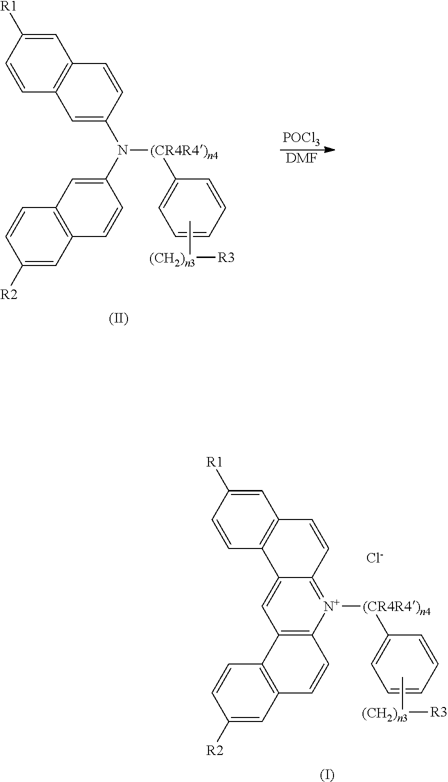

co-31: R₁═R₂═OMe, R₃═H; n3=n4=0;
co-330: R1═R2═OMe, R3═CO₂Me; n3=n4=0;
co-360: R1═R2═R3═OMe; n3=n4=0;
co-370: R1═R2═OMe, R3═NO₂; n3=n4=0;
co-38: R1═R2=R3═H; n3=n4=0;
co-39: R1═R2=R3═H, R4═R4′═H; n3=0, n4=1;
co-397: R1═R2═OMe, R3═H, R4═R4′═H; n3=0, n4=1;
co-398: R1═R2═R3═OMe, R4═R4′═H; n3=0, n4=1;
co-399: R1═R2═OMe, R3═H, R4═H, R4′═CH3; n3=0, n4=1.

General Procedure A: Synthesis of Dibenzoacridinium Compounds of Formula (I)

In a general procedure (according to scheme 1 above), precursor of Formula II (1 eq.) was solubilized in dry DMF and the mixture was cooled down to 0° C. Phosphorus oxychloride (2 eq.) was added dropwise under continuous stirring. The reaction mixture was allowed to warm up to room temperature and further heated up to 90° C. and stirred for 3 H. After removal of the solvent under vacuum, the crude product was dissolved in a mixture of dichloromethane and methanol (8:1). The target compound precipitated as a highly coloured solid by addition of ethyl acetate. The crude product was purified on silica gel column (CH₂Cl₂/MeOH (9:1, v/v)) to afford dibenzoacridinium compounds of Formula I as deeply coloured powders (from yellow to dark orange).

Synthesis of co-31: General procedure A was applied using precursor of Formula II-1 (2 g, 5 mmol, 1 eq.), dry DMF (30 mL) and POCl₃ (0.9 mL, 10 mmol, 2 eq.) to afford co-31 as a yellow powder (1.8 g, 4.1 mmol, 82% yield). ¹H NMR (300 MHz, dmso-d6): 6=11.22 (s, 1H), 9.66 (d, 2H, ³J_{H-H}=9.4 Hz), 8.50 (d, 2H, ³J_{H-H}=9.8 Hz), 7.98-7.96 (m, 3H), 7.88-7.85 (m, 2H), 7.77 (d, 2H, ⁴J_{H-H}=2.6 Hz), 7.70 (dd, 2H, ³J_{H-H}=9.4 Hz, ⁴J_{H-H}=2.6 Hz), 7.32 (d, 2H, ³J_{H-H}=9.8 Hz), 3.99 (s, 6H). ¹³C NMR (75 MHz, dmso-d6): 6=160.1, 139.9, 139.2, 137.8, 135.4, 132.0, 131.6, 131.3, 127.8, 126.7, 125.0, 122.1, 120.2, 117.5, 115.0, 110.1, 55.7. HR-MS ESI+(m/z): 416.1503 [M]+(calcd. 416.1645 for [C₂₉H₂₂NO₂]+).

Synthesis of co-360: General procedure A was applied using precursor of Formula II-2 (2.1 g, 5 mmol, 1 eq.), DMF (30 mL) and POCl₃ (0.9 mL, 10 mmol, 2 eq.) to afford co-360 as a yellow powder (1.4 g, 3.0 mmol, 61% yield). ¹H NMR (300 MHz, dmso-d6): δ=11.12 (s, 1H), 9.61 (d, 2H, ³J_{H-H}=9.3 Hz), 8.47 (d, 2H, ³J_{H-H}=9.7 Hz), 7.78 (d, 2H, ³J_{H-H}=9.0 Hz), 7.73 (d, 2H, ⁴J_{H-H}=2.7 Hz), 7.64 (dd, 2H, ³J_{H-H}=9.2 Hz, ⁴J_{H-H}=2.6 Hz), 7.49 (d, 2H, ³J_{H-H}=9.0 Hz), 7.38 (d, 2H, ³J_{H-H}=9.7 Hz), 4.00 (s, 3H), 3.97 (s, 6H). ¹³C NMR (75 MHz, dmso-d6): δ=161.0, 160.1, 140.4, 139.1, 135.3, 132.0, 130.3, 129.1, 126.8, 125.0, 122.1, 120.2, 117.7, 116.3, 110.1, 55.9, 55.8. HR-MS ESI+(m/z): 446.1761 [M]⁺ (calcd. 446.1751 for [C₃₀H₂₄NO₃]⁺).

Synthesis of co-330: General procedure A was applied using precursor of Formula II-3 (2.3 g, 5 mmol, 1 eq.), DMF (30 mL) and POCl₃ (0.9 mL, 10 mmol, 2 eq.) to afford co-330 as a yellow powder (1.7 g, 3.4 mmol, 68% yield). ¹H NMR (300 MHz, dmso-d6): δ=11.23 (s, 1H), 9.67 (d, 2H, ³J_{H-H}=9.3 Hz), 8.51 (d, 2H, ³J_{H-H}=8.6 Hz), 8.48 (d, 2H, ³J_{H-H}=9.5 Hz), 8.04 (d, 2H, ³J_{H-H}=8.6 Hz), 7.79 (d, 2H, ⁴J_{H-H}=2.6 Hz), 7.70 (dd, 2H, ³J_{H-H}=9.2 Hz, ⁴J_{H-H}=2.6 Hz), 7.36 (d, 2H, ³J_{H-H}=9.7 Hz), 4.02 (s, 3H), 3.99 (s, 6H). ¹³C NMR (75 MHz, dmso-d6): δ=165.4, 160.2, 156.2, 141.5, 139.8, 139.4, 132.5, 132.2, 132.1, 128.6, 126.8, 125.0, 122.1, 120.3, 117.6, 110.3, 55.8, 52.8. HR-MS ESI+(m/z): 474.1694 [M]⁺ (calcd. 474.1705 for [C₃₁H₂₄NO₄]⁺).

Synthesis of co-370: General procedure A was applied using precursor of Formula II-4 (2.2 g, 5 mmol, 1 eq.), DMF (30 mL) and POCl₃ (0.9 mL, 10 mmol, 2 eq.) to afford co-370 as an orange powder (1.9 g, 3.9 mmol, 78% yield). ¹H NMR (300 MHz, dmso-d6): δ=11.33 (s, 1H), 9.71 (d, 2H, ³J_{H-H}=9.3 Hz), 8.83 (d, 2H, ³J_{H-H}=9.0 Hz), 8.52 (d, 2H, ³J_{H-H}=9.6 Hz), 8.19 (d, 2H, ³J_{H-H}=9.0 Hz), 7.86 (d, 2H, ⁴J_{H-H}=2.6 Hz), 7.76 (dd, 2H, ³J_{H-H}=9.2 Hz, ⁴J_{H-H}=2.6 Hz), 7.44 (d, 2H, ³J_{H-H}=9.6 Hz), 4.02 (s, 6H). ¹³C NMR (75 MHz, dmso-d6): δ=160.3, 149.3, 142.8, 139.8, 139.5, 136.1, 132.1, 129.9, 126.8, 125.1, 122.1, 120.5, 117.6, 110.3, 55.8. HR-MS ESI+(m/z): 461.1496 [M]⁺ (calcd. 461.1496 for [C₂₉H₂₁N₂O₄]⁺).

Synthesis of co-38: General procedure A was applied using precursor of Formula II-5 (1.38 g, 4 mmol, 1 eq.), dry DMF (20 mL) and POCl₃ (0.73 mL, 8 mmol, 2 eq.) to afford co-38 as a yellow powder (1.4 g, 3.6 mmol, 92% yield). ¹H NMR (300 MHz, dmso-d6): δ=11.55 (s, 1H), 9.85 (d, 2H, ³J_{H-H}=8.3 Hz), 8.64 (d, 2H, ³J_{H-H}=9.6 Hz), 8.34 (d, 2H, ³J_{H-H}=7.3 Hz), 8.19 (t, 2H, ³J_{H-H}=7.2 Hz), 8.06-7.97 (m, 5H), 7.89 (m, 2H), 7.37 (d, 2H, ³J_{H-H}=9.6 Hz). ¹³C NMR (75 MHz, dmso-d6): δ=141.6, 140.2, 137.8, 137.3, 131.7, 131.3, 130.5, 130.1, 129.9, 129.8, 128.3, 127.8, 125.1, 124.8, 117.11. HR-MS ESI+(m/z): 356.1451 [M]⁺ (calcd. 356.1439 for [C₂₇H₁₈N]⁺).

Synthesis of co-39: General procedure A was applied using precursor of Formula II-6 (540 mg, 1.5 mmol, 1 eq.), dry DMF (8 mL) and POCl₃ (0.275 mL, 3 mmol, 2 eq.) to afford co-39 as a yellow powder (550 mg, 1.35 mmol, 90% yield). ¹H NMR (300 MHz, dmso-d6): δ=11.43 (s, 1H), 9.78 (d, 2H, ³J_{H-H}=8.3 Hz), 8.78 (d, 2H, ³J_{H-H}=9.7 Hz), 8.49 (d, 2H, ³J_{H-H}=9.7 Hz), 8.34 (d, 2H, ³J_{H-H}=7.9 Hz), 8.14 (t, 2H, ³J_{H-H}=8.2 Hz), 8.01 (t, 2H, ³J_{H-H}=7.8 Hz), 7.35 (m, 3H), 7.24 (m, 2H), 6.88 (s, 2H). ¹³C NMR (75 MHz, dmso-d6): δ=141.4, 140.8, 137.3, 134.4, 130.4, 130.1, 129.9, 129.6, 129.1, 128.8, 128.1, 126.0, 125.2, 125.1, 116.6, 54.5. HR-MS ESI+(m/z): 370.1601 [M]⁺ (calcd. 370.1590 for [C₂₉H₂₂NO₂]⁺).

Synthesis of co-397: General procedure A was applied using precursor of Formula II-7 (0.75 g, 1.8 mmol, 1 eq.), dry DMF (10 mL) and POCl₃ (0.33 mL, 3.6 mmol, 2 eq.) to afford co-397 as an orange powder (0.75 g, 1.6 mmol, 89% yield). ¹H NMR (300 MHz, dmso-d6): δ=11.16 (s, 1H), 9.63 (d, 2H, ³J_{H-H}=9.2 Hz), 8.63 (d, 2H, ³J_{H-H}=9.8 Hz), 8.44 (d, 2H, ³J_{H-H}=9.6 Hz), 7.78 (d, 2H, ⁴J_{H-H}=2.3 Hz), 7.69 (dd, 2H, ³J_{H-H}=9.1 Hz, ⁴J_{H-H}=2.6 Hz), 7.36 (m, 3H), 7.21 (d, 2H, ³J_{H-H}=8.8 Hz), 6.83 (s, 2H), 4.00 (s, 6H). ¹³C NMR (75 MHz, CD₂Cl₂/MeOD): δ=161.3, 140.8, 139.9, 134.7, 133.3, 132.5, 129.9, 129.3, 126.2, 126.0, 125.7, 122.7, 121.4, 116.4, 110.2, 56.1, 55.7. HR-MS ESI+(m/z): 430.1798 [M]⁺ (calcd. 430.1801 for [C₃₀H₂₄NO₂]⁺).

Synthesis of co-398: General procedure A was applied using precursor of Formula II-8 (252 mg, 0.56 mmol, 1 eq.), dry DMF (5 mL) and POCl₃ (0.1 mL, 1.12 mmol, 2 eq.) to afford co-398 as a yellow powder (260 mg, 0.53 mmol, 95% yield). ¹H NMR (300 MHz, dmso-d6): δ=11.21 (s, 1H), 9.65 (d, 2H, ³J_{H-H}=9.2 Hz), 8.66 (d, 2H, ³J_{H-H}=9.8 Hz), 8.48 (d, 2H, ³J_{H-H}=9.6 Hz), 7.82 (d, 2H, ⁴J_{H-H}=2.6 Hz), 7.73 (dd, 2H, ³J_{H-H}=9.1 Hz, ⁴J_{H-H}=2.6 Hz), 7.17 (d, 2H, ³J_{H-H}=8.8 Hz), 6.92 (d, 2H, ³J_{H-H}=8.8 Hz), 6.75 (s, 2H), 4.03 (s, 6H), 3.71 (s, 3H). ¹³C NMR (75 MHz, CD₂Cl₂/MeOD): δ=160.9, 160.0, 140.4, 139.4, 134.3, 132.1, 127.1, 126.0, 124.8, 122.3, 121.1, 116.2, 114.8, 109.86, 55.8, 55.3. HR-MS ESI+(m/z): 460.1904 [M]⁺ (calcd. 460.1907 for [C₃₁H₂₆NO₃]⁺).

Synthesis of co-399: General procedure A was applied using precursor of Formula II-9 (135 g, 0.3 mmol, 1 eq.), dry DMF (3 mL) and POCl₃ (0.3 mL, 0.6 mmol, 2 eq.). The reaction was carried out at RT to afford co-399 as a yellow powder (140 mg, 0.29 mmol, 94% yield). ¹H NMR (300 MHz, CD₂Cl₂/MeOD): δ=10.91 (s, 1H), 9.15 (d, 2H, ³J_{H-H}=9.2 Hz), 8.34 (m, 2H), 8.18 (m, 2H), 7.68 (dd, 2H, ³J_{H-H}=9.1 Hz, ⁴J_{H-H}=2.6 Hz), 7.52-7.39 (m, 6H), 7.19 (m, 2H), 4.03 (s, 6H), 2.43 (d, 3H, ³J_{H-H}=7.0 Hz). ¹³C NMR (75 MHz, dmso-d6): δ=160.1, 156.3, 139.3, 135.8, 131.7, 129.2, 127.9, 126.9, 126.0, 125.5, 122.7, 120.1, 117.8, 109.9, 62.2, 55.8, 18.23. HR-MS ESI+(m/z): 444.1950 [M]⁺ (calcd. 444.1958 for [C₃₁H₂₆NO₂]⁺).

General Procedure B (Buchwald-Hartwig Cross-Coupling Reaction): Synthesis of Arylamine Precursors of Formula II:

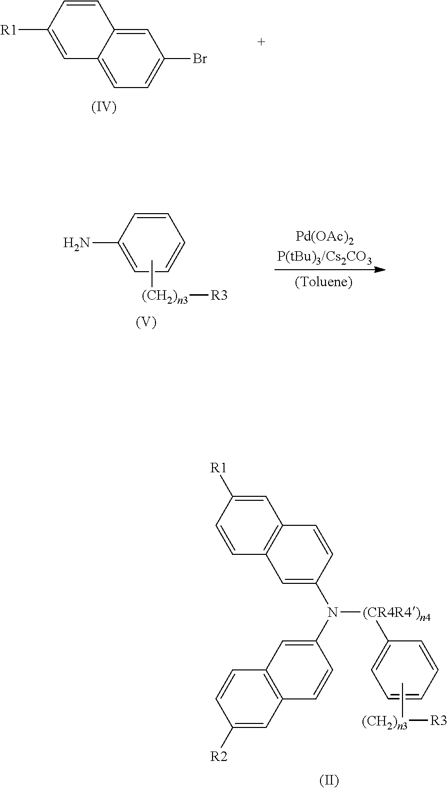

II-1: R₁═R₂═OMe, R₃═H; n3=n4=0;

II-2: R1═R2═OMe, R3═CO₂Me; n3=n4=0;

II-3: R1═R2═R3═OMe; n3=n4=0;

II-4: R1═R2═OMe, R3═NO₂; n3=n4=0;

II-5: R1═R2═R3═H; n3=n4=0.

In dry and degassed toluene were introduced Pd(OAc)₂ (0.06 equiv.) and P(t-Bu)₃ (0.12 equiv.). After 15 min of stirring, compound of Formula V or Formula III (1 equiv.), compound of Formula IV or Formula IV′ (4 equiv.) and Cs₂CO₃ (3 equiv.) were added successively. The solution was refluxed three days, cooled down to RT and diluted with CH₂Cl₂. The crude mixture was filtered, evaporated to dryness and purified on silica gel column (cyclohexane/CH₂Cl₂ 8:2, v/v) to afford compounds of Formula II as a coloured powders (from pale yellow to red).

Synthesis of II-1: General procedure B was applied using toluene (100 mL), Pd(OAc)₂ (148 mg, 0.66 mmol, 6%), P(t-Bu)₃ (0.32 mL, 1.3 mmol, 12%), aniline (1 mL, 11 mmol, 1 equiv.), 2-bromo-6-methoxynaphthalene (10.4 g, 44 mmol, 4 equiv.) and Cs₂CO₃ (10.7 g, 33 mmol, 3 equiv.) to afford II-1 as a pale yellow powder (2.7 g, 6.6 mmol, 60% yield). ¹H NMR (300 MHz, CDCl₃): δ=7.76 (d, 2H, ³J_{H-H}=9.0 Hz), 7.63 (d, 2H, ³J_{H-H}=9.0 Hz), 7.41 (s, 2H), 7.28-7.20 (m, 6H), 7.09-7.01 (m, 5H), 3.84 (s, 6H). ¹³C NMR (75 MHz, CDCl₃): δ=157.6, 132.9, 129.8, 129.6, 129.3, 129.1, 127.4, 126.6, 126.3, 123.4, 119.6, 118.4, 116.2, 106.0, 55.2. HR-MS ESI+(m/z): 405.1729 [M]⁺ (calcd. 405.1729 for [C₂₈H₂₃NO₂]⁺).

Synthesis of II-2: General procedure B was applied using toluene (100 mL), Pd(OAc)₂ (135 mg, 0.6 mmol, 6%), P(t-Bu)₃ (0.3 mL, 1.2 mmol, 12%), methyl 4-aminobenzoate (1.5 g, 10 mmol, 1 equiv.), 2-bromo-6-methoxynaphthalene (9.5 g, 40 mmol, 4 equiv.) and Cs₂CO₃ (9.7 g, 30 mmol, 3 equiv.) to afford II-2 as an orange powder (2.8 g, 6.1 mmol, 61% yield). ¹H NMR (300 MHz, CDCl₃): δ=7.83 (d, 2H, ³J_{H-H}=9.1 Hz), 7.80 (d, 2H, ³J_{H-H}=9.0 Hz), 7.71 (d, 2H, ³J_{H-H}=9.1 Hz), 7.62 (d, 2H, ⁴J_{H-H}=2.2 Hz), 7.32-7.28 (m, 4H), 7.12 (dd, 2H, ³J_{H-H}=9.0 Hz, ⁴J_{H-H}=2.5 Hz), 6.93 (d, 2H, ³J_{H-H}=9.0 Hz), 3.86 (s, 6H), 3.78 (s, 3H). ¹³C NMR (75 MHz, CDCl₃): δ=165.9, 157.1, 151.9, 141.5, 131.9, 130.8, 129.3, 128.8, 128.5, 125.7, 123.4, 120.7, 119.0, 118.5, 106.0, 55.2, 51.6. HR-MS ESI+(m/z): 486.1655 [M+Na]⁺ (calcd. 486.1676 for [C₃₀H₂₅NO₄Na]⁺).

Synthesis of II-3: General procedure B was applied using toluene (100 mL), Pd(OAc)₂ (164 mg, 0.7 mmol, 6%), P(t-Bu)₃ (0.35 mL, 1.5 mmol, 12%), p-anisidine (1.5 g, 12.2 mmol, 1 equiv.), 2-bromo-6-methoxynaphthalene (11.5 g, 48.8 mmol, 4 equiv.) and Cs₂CO₃ (11.9 g, 36.6 mmol, 3 equiv.) to afford II-3 as a pale yellow powder (3.1 g, 7.1 mmol, 58% yield). ¹H NMR (300 MHz, CDCl₃): δ=7.72 (d, 2H, ³J_{H-H}=9.0 Hz), 7.58 (d, 2H, ³J_{H-H}=9.0 Hz), 7.29 (d, 2H, ⁴J_{H-H}=2.3 Hz), 7.24 (d, 2H, ⁴J_{H-H}=2.3 Hz), 7.18 (dd, 2H, ³J_{H-H}=8.8 Hz, ⁴J_{H-H}=2.2 Hz), 7.06 (d, 2H, ³J_{H-H}=8.9 Hz), 7.05 (d, 2H, ³J_{H-H}=9.0 Hz), 6.93 (d, 2H, ³J_{H-H}=9.0 Hz), 3.83 (s, 6H), 3.75 (s, 3H). ¹³C NMR (75 MHz, CDCl₃): δ=156.8, 156.0, 144.3, 141.3, 130.8, 129.9, 128.4, 127.7, 126.7, 124.7, 119.6, 118.9, 114.8, 106.0, 55.6, 55.4. HR-MS ESI+(m/z): 435.1831 [M]⁺ (calcd. 435.1834 for [C₂₉H₂₅NO₃]⁺).

Synthesis of II-4: General procedure B was applied using toluene (100 mL), Pd(OAc)₂ (135 mg, 0.6 mmol, 6%), P(t-Bu)₃ (0.3 mL, 1.2 mmol, 12%), 4-nitroaniline (1.4 g, 10 mmol, 1 equiv.), 2-bromo-6-methoxynaphthalene (9.5 g, 40 mmol, 4 equiv.) and Cs₂CO₃ (9.7 g, 30 mmol, 3 equiv.) to afford II-4 as a red powder (3.6 g, 8.0 mmol, 80% yield). ¹H NMR (300 MHz, CDCl₃): δ=8.07 (d, 2H, ³J_{H-H}=9.4 Hz), 7.90 (d, 2H, ³J_{H-H}=8.9 Hz), 7.78 (d, 2H, ³J_{H-H}=8.9 Hz), 7.77 (d, 2H, ⁴J_{H-H}=2.2 Hz), 7.38 (dd, 2H, ³J_{H-H}=8.9 Hz, ⁴J_{H-H}=2.3 Hz), 7.36 (d, 2H, ⁴J_{H-H}=2.6 Hz), 7.16 (dd, 2H, ³J_{H-H}=9.0 Hz, ⁴J_{H-H}=2.5 Hz), 6.89 (d, 2H, ³J_{H-H}=9.4 Hz), 3.87 (s, 6H). ¹³C NMR (75 MHz, CDCl₃): δ=157.5, 153.6, 140.5, 138.9, 132.5, 129.2, 129.0, 128.8, 125.9, 125.7, 124.5, 119.2, 116.9, 106.0, 55.2. HR-MS ESI+(m/z): 473.1485 [M+Na]⁺ (calcd. 4737.1472 for [C₂₈H₂₂N₂O₄Na]⁺).

Synthesis of II-5: General procedure B was applied using toluene (100 mL), Pd(OAc)₂ (135 mg, 0.6 mmol, 6%), P(t-Bu)₃ (0.3 mL, 1.2 mmol, 12%), aniline (0.93 g, 10 mmol, 1 equiv.), 2-bromo-naphthalene (8.3 g, 40 mmol, 4 equiv.) and Cs₂CO₃ (9.7 g, 30 mmol, 3 equiv.) to afford II-5 as a pale yellow powder (3 g, 8.7 mmol, 87% yield). ¹H NMR (300 MHz, CDCl₃): δ=7.80 (d, 2H, ³J_{H-H}=7.1 Hz), 7.77 (d, 2H, ³J_{H-H}=9.1 Hz), 7.61 (d, 2H, ³J_{H-H}=7.1 Hz), 7.50 (d, 2H, ⁴J_{H-H}=2.2 Hz), 7.46-7.29 (m, 8H), 7.21 (d, 2H, ³J_{H-H}=8.7 Hz), 7.11 (t, 1H, ³J_{H-H}=7.2 Hz). ¹³C NMR (75 MHz, CDCl₃): δ=147.8, 145.5, 134.5, 130.3, 129.4, 129.0, 127.7, 127.1, 126.4, 124.8, 124.7, 124.6, 123.2, 120.6. HR-MS ESI+(m/z): 345.1514 [M+]⁺ (calcd. 345.1517 for [C₂₆H₁₉N]⁺).

Synthesis of II-6:

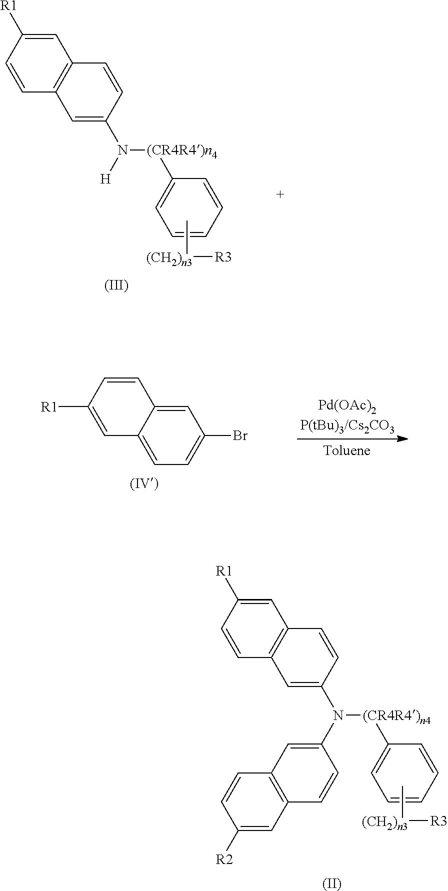

II-6: R1═R2═R3═H; R4═R4′═H, n3=0, n4=1

II-7: R1═R2═OMe, R3═H, R4═R4′═H, n3=0, n4=1

II-8: R1═R2═R3═OMe, R4═R4′═H, n3=0, n4=1

II-9: R1═R2═OMe, R3═H, R4═H, R4′=Me, n3=0, n4=1.

General procedure B was applied using toluene (50 mL), Pd(OAc)₂ (47 mg, 0.21 mmol, 6%), P(t-Bu)₃ (101 μL, 0.42 mmol, 12%), precursor of Formula III-1 (0.84 g, 3.6 mmol, 1 equiv.), 2-bromo-naphthalene (1.5 g, 7.2 mmol, 2 equiv.) and Cs₂CO₃ (2.35 g, 7.2 mmol, 2 equiv.) to afford II-6 as a pale yellow powder (0.58 g, 1.6 mmol, 44% yield). ¹H NMR (300 MHz, CDCl₃): δ=7.79-7.74 (m, 4H), 7.66 (d, 2H, ³J_{H-H}=8.1 Hz), 7.50-7.26 (m, 13H), 5.28 (s, 2H). ¹³C NMR (75 MHz, CDCl₃): δ=145.6, 138.9, 134.6, 129.4, 128.9, 128.6, 127.5, 126.9, 126.8, 126.6, 126.3, 124.0, 122.2, 116.3, 56.6. HR-MS ESI+(m/z): 359.1675 [M]⁺ (calcd. 359.1674 for [C₂₇H₂₁N]⁺).

Synthesis of II-7: General procedure B was applied using toluene (30 mL), Pd(OAc)₂ (36 mg, 0.16 mmol, 6%), P(t-Bu)₃ (77 μL, 0.32 mmol, 12%), precursor of Formula III-2 (0.70 g, 2.66 mmol, 1 equiv.), 2-bromo-6-methoxynaphthalene (1.26 g, 5.32 mmol, 2 equiv.) and Cs₂CO₃ (1.73 g, 5.32 mmol, 2 equiv.) to afford II-7 as a pale yellow powder (0.83 g, 1.98 mmol, 75% yield). ¹H NMR (300 MHz, CDCl₃): δ=7.62 (d, 2H, ³J_{H-H}=8.9 Hz), 7.52 (d, 2H, ³J_{H-H}=9.6 Hz), 7.45-7.39 (m, 4H), 7.34-7.21 (m, 5H), 7.10-7.07 (m, 4H), 5.17 (s, 2H), 3.89 (s, 6H). ¹³C NMR (75 MHz, CDCl₃): δ=156.6, 143.9, 138.9, 130.4, 129.9, 128.6, 128.5, 127.7, 126.9, 122.7, 119.0, 116.7, 105.9, 57.0, 55.4. HR-MS ESI+(m/z): 419.1877 [M]+(calcd. 419.1885 for [C₂₉H₂₅N]⁺).

Synthesis of II-8: General procedure B was applied using toluene (15 mL), Pd(OAc)₂ (13.5 mg, 0.06 mmol, 6%), P(t-Bu)₃ (30 mL, 0.12 mmol, 12%), precursor of Formula III-3 (293 mg, 1 mmol, 1 equiv.), 2-bromo-6-methoxynaphthalene (475 mg, 2 mmol, 2 equiv.) and Cs₂CO₃ (650 mg, 2 mmol, 2 equiv.) to afford II-8 as a pale yellow powder (267 mg, 0.67 mmol, 60% yield). ¹H NMR (300 MHz, CDCl₃): δ=7.61 (d, 2H, ³J_{H-H}=8.8 Hz), 7.54 (d, 2H, ³J_{H-H}=9.8 Hz), 7.43 (d, 2H, ⁴J_{H-H}=2.2 Hz), 7.35-7.30 (m, 4H), 7.11-7.07 (m, 4H), 6.80 (d, 2H, ³J_{H-H}=8.7 Hz), 5.11 (s, 2H), 3.90 (s, 6H), 3.76 (s, 3H). ¹³C NMR (75 MHz, CDCl₃): δ=158.6, 156.6, 143.7, 130.4, 129.8, 128.4, 128.3, 127.7, 122.7, 118.9, 116.9, 114.4, 114.0, 105.8, 56.5, 55.3, 55.2. HR-MS ESI+(m/z): 450.2056 [M+H]⁺ (calcd. 450.2069 for [C₃₀H₂₈NO₃]⁺).

Synthesis of II-9: General procedure B was applied using toluene (30 mL), Pd(OAc)₂ (32 mg, 0.14 mmol, 6%), P(t-Bu)₃ (67 mL, 0.28 mmol, 12%), precursor of Formula III-4 (665 mg, 2.4 mmol, 1 equiv.), 2-bromo-6-methoxynaphthalene (1.1 g, 4.8 mmol, 2 equiv.) and Cs₂CO₃ (1.6 g, 4.8 mmol, 2 equiv.) to afford II-9 as a pale yellow powder (364 mg, 0.84 mmol, 35% yield). ¹H NMR (300 MHz, CDCl₃): δ=7.58-7.49 (m, 6H), 7.39 (d, 2H, ⁴J_{H-H}=1.8 Hz), 7.31-7.21 (m, 3H), 7.14 (dd, 2H, ³J_{H-H}=8.8 Hz, ⁴J_{H-H}=2.2 Hz), 7.10-7.06 (m, 4H), 5.48 (q, 1H, ³J_{H-H}=6.9 Hz), 3.89 (s, 6H), 1.63 (d, 3H, ³J_{H-H}=6.9 Hz). ¹³C NMR (75 MHz, CDCl₃): δ=156.8, 143.2, 142.6, 130.7, 129.6, 128.7, 128.4, 127.5, 127.4, 127.1, 124.5, 119.5, 118.9, 105.75, 59.3, 55.3, 19.5. HR-MS ESI+(m/z): 434.2108 [M+H]⁺ (calcd. 434.2120 for [C₃₀H₂₈NO₂]⁺).

General Procedure C (Ullman Coupling Reaction): Synthesis of Arylamine Precursors of Formula III:

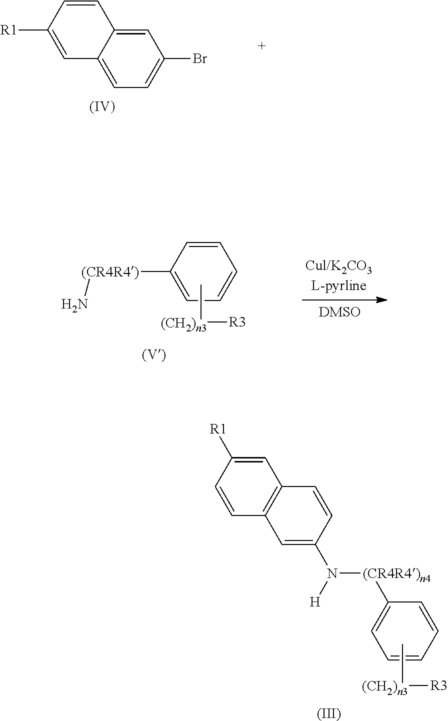

III-1: R1═R3═H; R4═R4′═H, n3=0, n4=1

III-2: R1═OMe, R3═H, R4═R4′═H, n3=0, n4=1

III-3: R1═R3═OMe, R4═R4′═H, n3=0, n4=1

III-4: R1═OMe, R3═H, R4═H, R4′=Me, n3=0, n4=1.

A mixture of compound of Formula IV (1 equiv.), compound of Formula V′ (1.5 equiv.), K₂CO₃ (2 equiv.), CuI (0.1 equiv.) and L-proline (0.2 equiv.) in DMSO (3 mL) was stirred at 70° C. for 24 H. The mixture was poured into water and the aqueous layer was extracted with ethyl acetate. The combined organic layers were washed with brine, water, dried over MgSO₄ and evaporated to dryness. The crude was purified on silica gel column (cyclohexane/EtOAc 8:2, v/v) to afford compounds of Formula III as a colourless powders.

Synthesis of III-1: General procedure C was applied using 2-bromo-naphthalene (1 g, 5 mmol, 1 equiv.), benzylamine (803 mg, 7.5 mmol, 1.5 equiv.), K₂CO₃ (1.38 g, 10 mmol, 2 equiv.), CuI (95 mg, 0.5 mmol, 0.1 equiv.) and L-proline (115 mg, 1 mmol, 0.2 equiv.) in DMSO (3 mL) to afford III-1 as a pale yellow powder (850 g, 3.6 mmol, 72% yield ¹H NMR (300 MHz, CDCl₃): δ=7.55 (d, 1H, ³J_{H-H}=8.8 Hz), 7.52 (d, 1H, ³J_{H-H}=8.9 Hz), 7.43-7.28 (m, 5H), 7.07-7.02 (m, 2H), 6.94 (d, 1H, ³J_{H-H}=8.7 Hz), 6.90 (s, 1H), 4.41 (s, 2H), 3.88 (s, 6H). ¹³C NMR (75 MHz, CDCl₃): δ=144.7, 140.5, 130.6, 129.5, 128.8, 127.6, 127.3, 125.5, 119.6, 118.5, 105.3, 104.2, 49.0. HR-MS ESI+(m/z): 234.1280 [M+H]⁺ (calcd. 234.1283 for [C₁₇H₁₆N]⁺).

Synthesis of III-2: General procedure C was applied using benzylamine (803 mg, 7.5 mmol, 1.5 equiv.), 2-bromo-6-methoxynaphthalene (1.18 g, 5 mmol, 1 equiv.), K₂CO₃ (1.38 g, 10 mmol, 2 equiv.), CuI (95 mg, 0.5 mmol, 0.1 equiv.) and L-proline (115 mg, 1 mmol, 0.2 equiv.) in DMSO (3 mL) to afford 111-2 as a pale yellow powder (800 mg, 3.0 mmol, 61% yield). ¹H NMR (300 MHz, CDCl₃): δ=7.55 (d, 1H, ³J_{H-H}=8.8 Hz), 7.52 (d, 1H, ³J_{H-H}=8.9 Hz), 7.43-7.28 (m, 5H), 7.07-7.02 (m, 2H), 6.94 (d, 1H, ³J_{H-H}=8.7 Hz), 6.90 (s, 1H), 4.41 (s, 2H), 3.88 (s, 6H). ¹³C NMR (75 MHz, CDCl₃): δ=155.4, 143.8, 139.1, 130.4, 128.8, 128.6, 127.9, 127.7, 127.5, 118.9, 118.5, 106.3, 106.2, 55.4, 49.1. HR-MS ESI+(m/z): 264.1380 [M+H]⁺ (calcd. 264.1388 for [C₁₈H₁₈NO]⁺).

Synthesis of III-3: General procedure C was applied using 4-methoxybenzylamine (1.03 g, 7.5 mmol, 1.5 equiv.), 2-bromo-6-methoxynaphthalene (1.18 g, 5 mmol, 1 equiv.), K₂CO₃ (1.38 g, 10 mmol, 2 equiv.), CuI (95 mg, 0.5 mmol, 0.1 equiv.) and L-proline (115 mg, 1 mmol, 0.2 equiv.) in DMSO (3 mL) to afford 111-3 as a pale yellow powder (296 mg, 1 mmol, 20% yield). ¹H NMR (300 MHz, CDCl₃): δ=7.69-7.64 (m, 2H), 7.56-7.51 (m, 2H), 7.28-7.25 (m, 2H), 6.93 (dd, 1H, ³J_{H-H}=8.7 Hz, ⁴J_{H-H}=2.3 Hz), 6.80 (d, 1H, ³J_{H-H}=8.7 Hz), 6.68 (d, 2H, ³J_{H-H}=8.7 Hz), 4.86 (s, 1H), 4.36 (s, 2H), 3.87 (s, 3H), 3.63 (s, 3H). ¹³C NMR (75 MHz, CDCl₃): δ=155.6, 143.8, 139.8, 130.8, 128.8, 128.5, 127.8, 127.5, 127.3, 119.9, 118.8, 106.8, 106.0, 56.4, 55.6, 49.5. HR-MS ESI+(m/z): 294.1490 [M+H]⁺ (calcd. 294.1494 for [C₁₉H₂₀NO₂]⁺).

Synthesis of III-4: General procedure C was applied using (R)-(+)-□-methylbenzylamine (0.90 g, 7.5 mmol, 1.5 equiv.), 2-bromo-6-methoxynaphthalene (1.18 g, 5 mmol, 1 equiv.), K₂CO₃ (1.38 g, 10 mmol, 2 equiv.), CuI (95 mg, 0.5 mmol, 0.1 equiv.) and L-proline (115 mg, 1 mmol, 0.2 equiv.) in DMSO (3 mL) to afford 111-4 as a pale yellow powder (0.67 g, 2.5 mmol, 50% yield). ¹H NMR (300 MHz, CDCl₃): δ=7.48 (d, 1H, ³J_{H-H}=8.7 Hz), 7.42-7.38 (m, 3H), 7.33-7.29 (m, 2H), 7.26-7.20 (m, 2H), 7.01-6.97 (m, 2H), 6.89 (dd, 1H, ³J_{H-H}=8.7 Hz, ⁴J_{H-H}=2.3 Hz), 6.68 (s, 1H), 4.58 (q, 1H, ³J_{H-H}=6.7 Hz), 3.85 (s, 3H), 1.60 (d, 3H, ³J_{H-H}=6.7 Hz). ¹³C NMR (75 MHz, CDCl₃): δ=155.9, 142.7, 129.7, 129.5, 128.7, 128.4, 128.0, 127.7, 127.5, 126.6, 119.3, 118.9, 106.0, 56.4, 55.3, 23.6. HR-MS ESI+(m/z): 278.1532 [M+H]⁺ (calcd. 278.1545 for [C₁₉H₂₀NO]⁺).

II—Biological Activity

1. Evaluation of the Interaction Between the Ligands and the Viral G4s

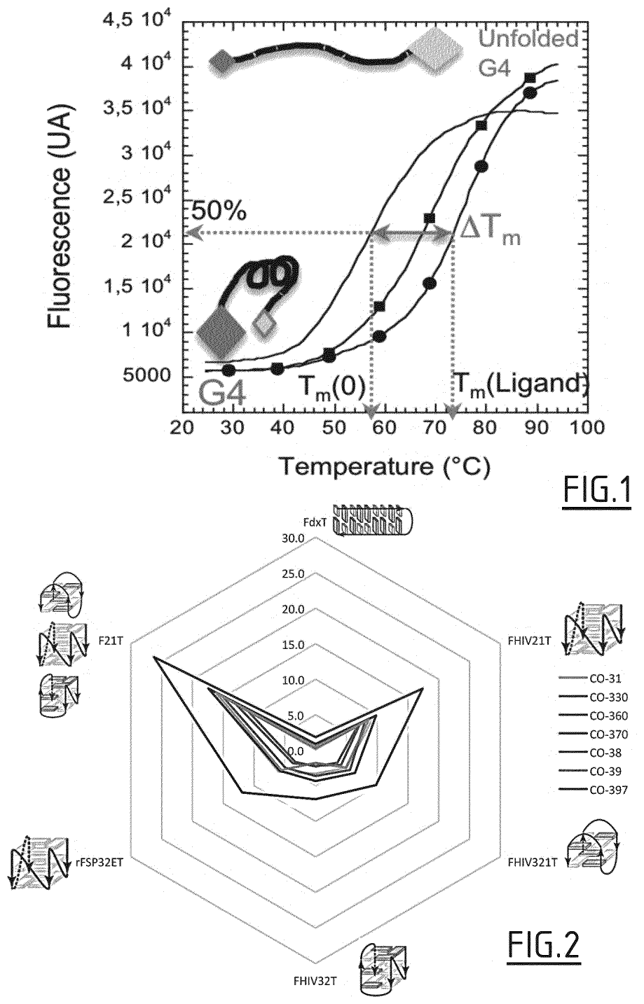

Using a FRET-based stabilization test, the ability of the compounds of the invention to bind to 5 viral G4s and 1 duplex control was evaluated. In this test, thermal denaturation experiments followed by fluorescence were performed. Ligand binding on the G4 stabilizes the structure and this results in an increase in the temperature of half-dissociation of the latter. The higher the ΔTm, the better is the affinity of the ligand for the target (FIGS. 1 and 2). In this test, CO397 showed the highest affinity for the G4s.

2. Effect on Native HIV Infectivity

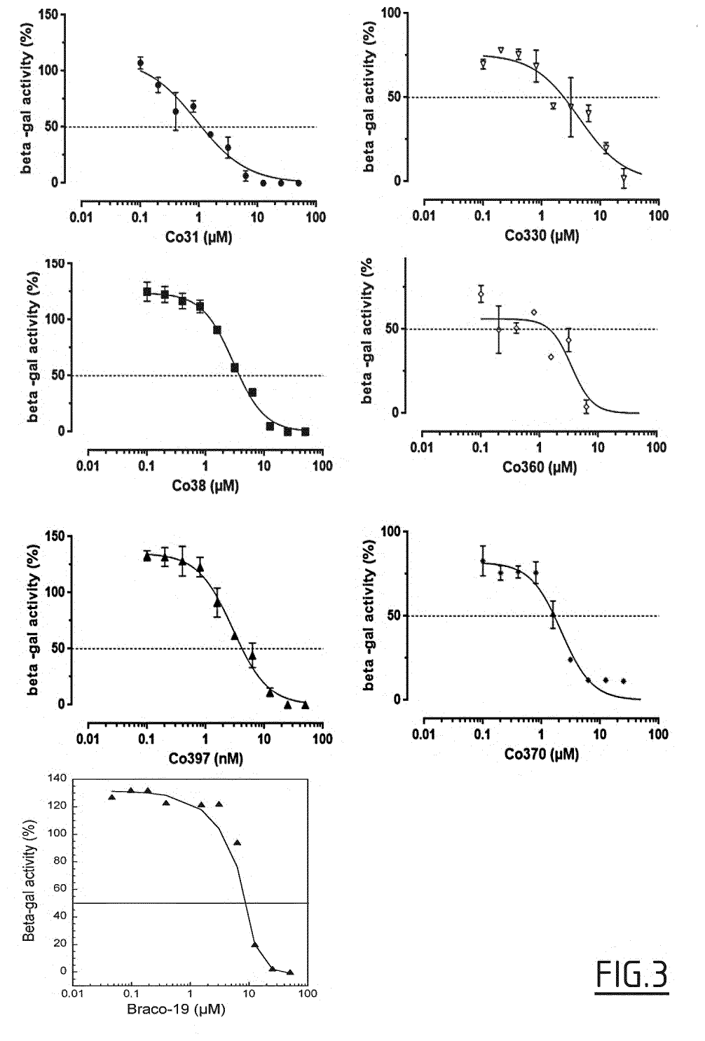

The study of the effect of the G4 ligands on HIV-1 infectivity was performed in a BSL-3 laboratory. For this infectivity assays HeLa P4 cells were used as cellular model of HIV-1 replication. It encodes a Tat-inducible β-galactosidase whose expression is linked to the expression of the viral Tat protein. All the compounds were able to reduce HIV-1 infectivity in the low micromolar concentrations (FIG. 3).

3. Effects of Acridinium Derivatives on Native Filovirus Infectivity

The goal was to determine the inhibitory effect induced by a new class of G4 ligands (acridinium compounds) on the infectivity of native Ebola or Marburg viruses. For this set of experiments, performed in the INSERM BSL4 laboratory Jean Mérieux in Lyon, a derivative called CO39 was selected.

Prior to the viral replication tests, the cytotoxic potency of each molecule was tested on VeroE6 cells. The purpose is to define the concentrations of molecules that can be used on the cells without inducing cytotoxicity. In a second step, the viral replication tests in the presence of the compounds were performed. The molecules were incubated at the pre-defined concentrations (5-20 μM) in the presence of Ebola or Marburg virus. After 7 days of incubation, the supernatant containing the newly produced viruses was harvested and divided in 2 parts in order to quantify the antiviral effect by 2 different approaches: i) In the 1^{st} approach, the supernatant was inactivated in the P4 laboratory, the viral RNA was then extracted in the P2 laboratory. The amount of specific RNA from Ebola or Marburg viruses in each sample was precisely quantified by qRT-PCR to determine the replication rate of the viruses. This approach has been implemented 2 times using 2 different stocks of viruses at 4 months interval 5 (September 2017 & January 2018). li) In the 2nd approach, the number of EBOLA or Marburg infectious particles were counted by immunohistochemistry method allowing to precisely quantify the number of infectious particles for each tested molecule.

Methods:

HIV-1:

HIV-1 production: HIV-1 was produced by co-culture of MT4 cells and chronically infected H9_{Lai} cells (0.5 10⁶ cells/ml each) for 48 h. Viral titer was evaluated by RT-qPCR quantification of viral RNA.

The molecules were dissolved in pure water at a concentration of 1 mM.

Cell lines: HeLa P4 cells (Charneau et al J Mol Biol 241:651-662) were used for infection experiments were maintained in DMEM medium (Invitrogen) supplemented with 10% inactivated fetal calf serum (FCS), 1 mg/ml geneticin (G418, Gibco-BRL). They encode a Tat-inducible β-galactosidase whose expression driven by the HIV-1 LTR is linked to the expression of the viral Tat protein. Hela P4 cells were seeded in a 96-wells plate containing 10000 cells per well 24 hours before infection. Serial dilutions of drogues were added on the cells at the time of infection.

Infectivity assays: After 24 hours of infection, the p-gal activity is quantified by adding 4-MUG mix (Tris-HCl 50 mM; pH 8; p-mercaptoethanol 100 mM; Triton X-100 0.05%; 4-MUG 5 mM) on the cells. Fluorescence associated with the reaction product was monitored 24 hours after adding the 4-MUG mix using a Cytofluor-II plate reader (Applied Biosystems, Foster City, CA) with excitation/emission filters at 360/460 nm.

Cytotoxicity study: Cytotoxicity effect of the molecules was performed in similar conditions without virus and measured with the CellTiter 96® AQueous One Solution Cell Proliferation Assay System (Promega).

Filovirus:

Cell line: VeroE6 cells from the Inserm Jean Mérieux P4 laboratory seeded in 96 well plates to 1.5 million cells per plate the day before each test to reach a confluence of 80% on the day of the test.

The molecules were dissolved in pure water at a concentration of 0.2 mM Cytotoxicity study: Each molecule was tested at final concentrations of 25 μM, 20 μM, 10 μMM and 5 μM on the VeroE6 cells. The molecules diluted in DMEM medium (supplemented with 2% of FCS and 1% of penistreptomycin) were injected on the cells and incubated for 7 days at 37° C. and 5% CO₂. After incubation, the cells were observed under a microscope and then a viability test was performed using a 10% resazurin solution. This viability test is read on a Tecan apparatus after 2 h incubation at 37° C. and resulting in a cell survival rate. The two cell plates were observed under a microscope: the control cells were validated (no change in cell morphology) and the cells were not different, except for the high concentration of AuPG molecule. Adding this molecule to the cells resulted in a marked increase in cell death.

Ebola Gabon and Marburg virus stocks production: Ebola Gabon virus stock was produced on VeroE6 cells (passage 7) with a final viral titer of 8.106 FFU/mLa. Stock of Marburg virus was produced on VeroE6 cells (passage 5) with a final viral titer of 9.17%-FFU/ml.

Infection of VeroE6 Cells by EBOV or MBGV.

Vero E6 cells are incubated with different concentrations of molecules and then infected with Ebola or Marburg viruses using the following optimal conditions of infection: The Ebola or Marburg viruses (one 96-well plate for Ebola Gabon and one 96-well plate for Marburg virus), diluted to an MOI of 0.001 were deposited at the same volume-to-volume onto VeroE6 cells at 80% confluence, followed by a 7-day incubation in the presence of the tested molecules at 37° C., 5% of C02. Virus controls have also been created. This involves replacing the molecule with water at the same concentration, which means that 2 Tv different (Tv1−Tv2) mimicked the 2 concentrations of molecules. After 7 days of incubation at 37° C., the supernatant containing the newly produced viruses in each well was harvested and was divided in 2 parts in order to quantify the antiviral effect by 2 different approaches: i) Quantification of EBOV or MBGV RNA copies by qRT-PCR and ii) Quantification of the infectious EBOV or MBGV particles by immunohistochemistry method.

Quantification of EBOV or MBGV RNA copies by qRT-PCR: In this 1^{st} approach, after incubation, the cells were observed under a microscope, then the first part of the supernatant of each well was inactivated by the method validated by the P4 Inserm laboratory (inactivation in AVL lysis buffer and absolute EtOH-kit “QIAmp mini viral RNA kit” from Qiagen). the viral RNA was then extracted in the P2 laboratory on a column (Qiagen's “QIAmp mini viral RNA kit” kit) using the QiaCube 96-well plate automaton. The extracted RNAs were then amplified and detected in duplicate by means of a qRT-PCR (Altona's RealStar filovirus screen RT PCR kit) on the LightCycler 480 (Roche). The amount of specific RNA from Ebola or Marburg viruses in each sample is precisely quantified by qRT-PCR to determine a possible inhibition of the multiplication of the virus. This approach has been conducted 2 times using 2 different stocks of viruses at 5 months interval.

Quantification of the infectious EBOV or MBGV particles by immunohistochemistry method: In the 2nd approach performed in the BSL4 laboratory, the number of EBOLA or Marburg infectious particles were counted in the second part of the supernatant. The supernatants were serially diluted and plated replated onto VeroE6 cells (1 plate with 24 wells per supernatant, 1 well per dilution). After 7 days of incubation at 37° C. and 5% CO₂, the cells were observed under a microscope and then fixed with formaldehyde. The infectious particles were revealed by immunohistochemistry method. The count of colored foci with Ebola- or Marburg-specific antibodies labeled with peroxidase allowed to precisely quantify the number of infectious particles for each tested molecule and the viral titer is calculated as FFU/ML (counting infectious virus particles only).

Results:

HIV-1:

Effect of Ligands on HIV-1 Infectivity and Cell Viability.

The effect of the G4 ligands on HIV-1 infectivity was evaluated in the BSL-3 facility of TBM-core (UB'L3, University of Bordeaux). Each molecule was tested at concentrations between 50 μM and 0.1 μM by employing serial two-fold dilutions. All the compounds but CO39 were able to reduce HIV-1 infectivity in the low micromolar concentrations with a clear dose response effect (FIG. 3). Complete inhibition was observed at 10 μM concentration for C031, C0370, C038, CO397.

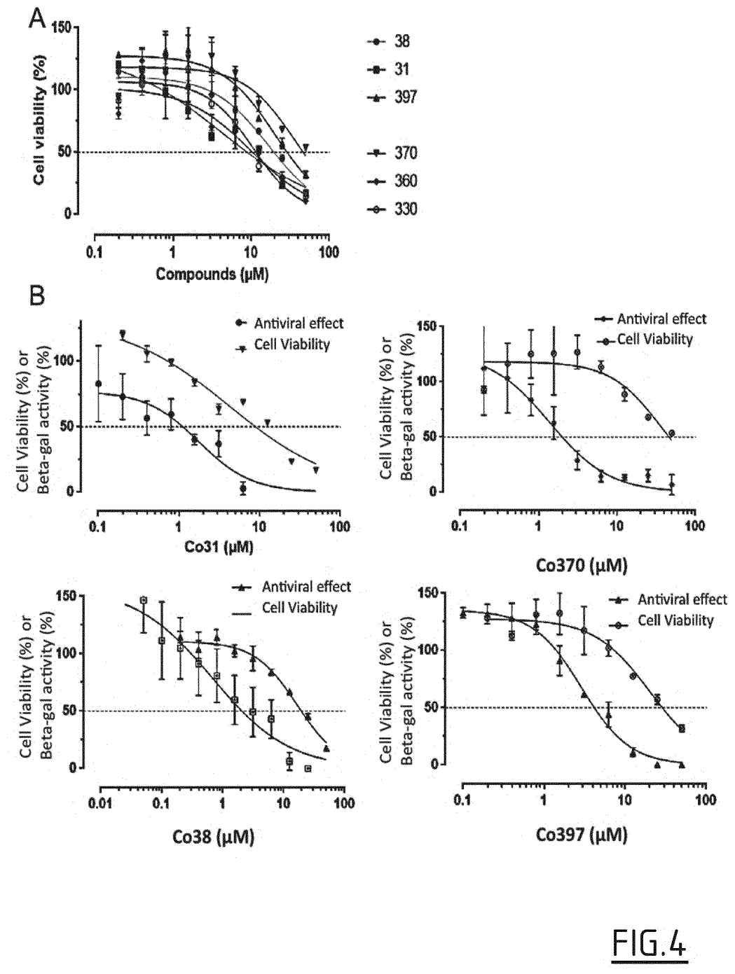

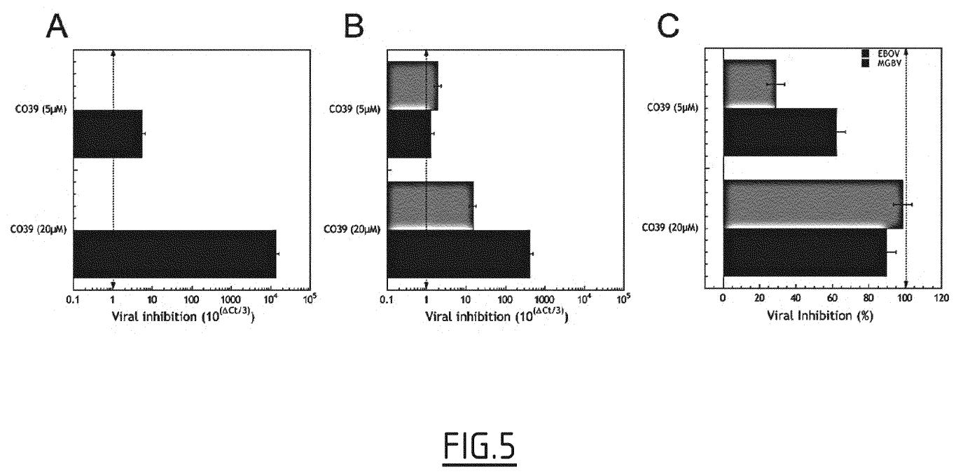

In a second step, the cytotoxic effect of all compounds was evaluated on HeLa P4 cells. All compounds showed a CC₅₀ superior to the IC₅₀ concentration. The best selectivity index (SI=17) was obtained for C0370 compound. Comparison between inhibition and cell viability is shown in FIG. 4.

Comparison with a Reference Compound

The activity of the compounds of the invention was compared with that of BRACO-19 disclosed in Nucleic Acids Research, 2018, 1-14 of formula:

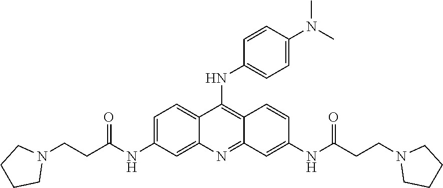 [SMILES: CN(C)c1ccc(Nc2c3ccc(NC(=O)CCN4CCCC4)cc3nc3cc(NC(=O)CCN4CCCC4)ccc23)cc1]

As apparent in FIG. 3, Braco19 inhibits the HIV-1 infectivity in the reporter cells with IC₅₀ around 8 μM. This value is slightly higher that the IC₅₀ for most acridinium compounds of the invention. In particular, C0370 and C031 are at least five time more active on HIV-1 than BRACO-19. This suggests that acridinium are more efficient than Braco19 in this model. In 2014, Braco19 was reported to inhibit HIV-1 infectivity in cell lines with IC₅₀ around 6 μM in MT4 cells. The antiviral effect of Braco19 was also confirmed in primary infected cells, while less efficient with respect to MT4 infected cell lines. In conclusion, the acridin compounds described in this study are more efficient than the well known G4 ligand Braco19.

Filovirus

Cytotoxicity Study:

The cells were observed under a microscope: the control cells were validated (no change in cell morphology) and the cells were not different, no toxicity was observed for CO39 compound.

Viral Inhibition Quantified by qRT-PCR:

The positive and negative controls of both qRT-CR were validated, as well as the amplification curves, which validates the experiment. The average Ct (average of duplicates) obtained for each sample was then compared to the average Ct of the corresponding virus control. The difference in Ct between the control and the sample was established for each condition tested in order to quantify the inhibition of the viral replication induced by each molecule. It is generally considered that a difference of 3 Ct is equivalent to a difference of 1 log (1 Ox) in terms of infectious viral replication. Thus, it can be estimated that a molecule which allows a reduction of 3 Ct induces the reduction of the number of Ebola Gabon or Marburg virus RNA copies by a factor of approximately 10 as represented in FIG. 4A and FIG. 4B.

Ebola virus: In the presence of 20 μM of CO39 infectivity was inhibited by a factor of 15 to 400. In the presence of 5 μM of CO39 infectivity was inhibited by a factor of 5.

Marburg virus: In the presence of 20 μM of CO39 infectivity was inhibited by a factor of 13 000. In the presence of 5 μM of CO39 infectivity was inhibited by a factor of 2. Notably, these experiments have been conducted 2 times using 2 different stocks of viruses at 5 months interval resulting in very similar inhibitions.

Viral Inhibition Quantified by Immunohistochemistry:

The infectious particles were revealed by immunohistochemistry method. The count of colored foci with Ebola- or Marburg-specific antibodies labeled with peroxidase allowed to precisely quantify the number of infectious particles for each tested molecule. The mean infectious viral titer (average of duplicates) obtained for each sample was then compared to the infectious viral titer of the corresponding virus control. The difference in titer between the control and the sample has been established for each condition tested, and makes it possible to quantify the inhibition of the viral replication linked to each molecule (FIG. 4C).

Ebola virus: Overall, The quantification of the inhibition by this technique is well correlated with the rt-QPCR approach and confirms the observed antiviral effects. Indeed the strongest inhibitions (almost 100%) were obtained for CO39 at 20 μM. Notably, a decrease of the effect was observed when lowering the concentrations to 5 μM (almost 60%) showing a clear dose effect in the inhibition.

Marburg virus: Overall, The quantification of the inhibition by this technique is well correlated with the RT-qPCR approach and confirms the observed antiviral effects. Indeed the strongest inhibitions (almost 100%) were obtained for CO39 at 20 μM. The lowest effects were obtained with CO39 at 5 μM (30%). Again, a decrease of the effect was observed when lowering the concentrations showing a clear dose effect in the inhibition.

### CONCLUSION

The activity of the two filoviruses was respectively divided by 5 to 5000 in the presence of 5 μM to 20 μM of CO39 acridinium derivatives without showing any cytotoxicity at this concentration. Notably, a decrease of the effect was observed when lowering the concentrations showing a clear dose effect in the inhibition. Furthermore, some of these experiments have been conducted 2 times using two different stocks of viruses at 5 months interval resulting in very similar inhibitions. The inhibitory effect was demonstrated by two different approaches.

1. A compound of formula (I): 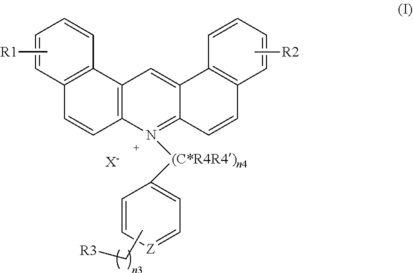
wherein
R1, R2 and R3 are identical or different and are located at any position of the benzene ring on which they are attached;
R1, R2 independently represent H, —O(C1-C6)alkyl OH, (C1-C6)alkyl, COOR, NO₂, CN, NRR′;
R3 is chosen from H, COO(C1-C6)alkyl, —O(C1-C6)alkyl, NO₂, (C1-C6)alkyl, OH, COOH, CN, NRR′, CF₃, NRR′R″⁺/Y⁻, or a guanidine group chosen from 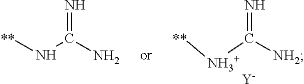
where ** illustrates the attachment of the guanidine group to the terminal carbon of the —(CH₂)ₙ₃— chain;
C* denotes an optionally asymmetric carbon atom;
R4 and R4′ are identical or different and independently represent H, (C1-C6)alkyl;
n3 and n4 are identical or different and are independently chosen from an integer from 0 to 6 inclusive;
Z represents CH, N, C or N⁺/X′⁻ when R3 is not H;
R, R′ and R″ are identical or different and independently represent H, (C1-C6)alkyl;
X⁻, X′⁻ and Y⁻ independently represent an anion;
optionally in the form of a hydrate or of a solvate thereof.

2. A compound according to claim 1 which is of formula (IA): 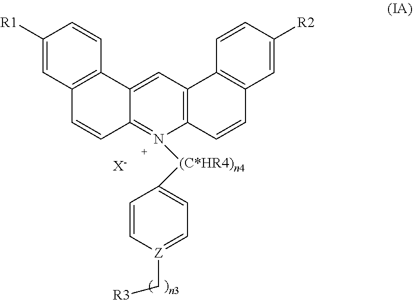
wherein R1, R2, R3, R4, X, Z, n3 are defined as in claim 1; and n4 is 0 or 1.

3. A compound according to claim 1 wherein:
R1, R2 independently represent H, —O(C1-C6)Alkyl;
R3 is chosen from H, COO(C1-C6)Alkyl, —O(C1-C6)Alkyl, NO₂;
R4 represents H or (C1-C6)Alkyl and R4′ represents H;
n4 is 0 or 1;
n3 is 0;
R, R′ and R″, identical or different, independently represent H, (C1-C6)alkyl;
X⁻ represents a halide; and
Z represents CH or N, or C or N⁺/X′⁻ when R3 is not H.

4. A compound according to claim 1 which is chosen from the following group of compounds: 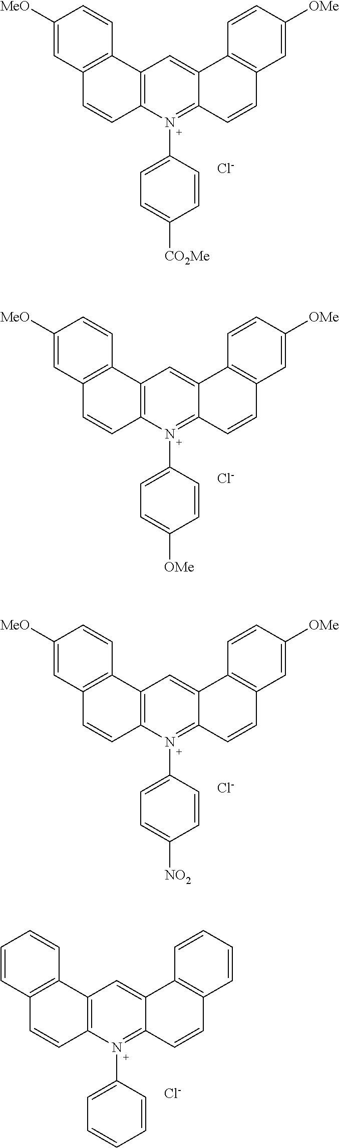 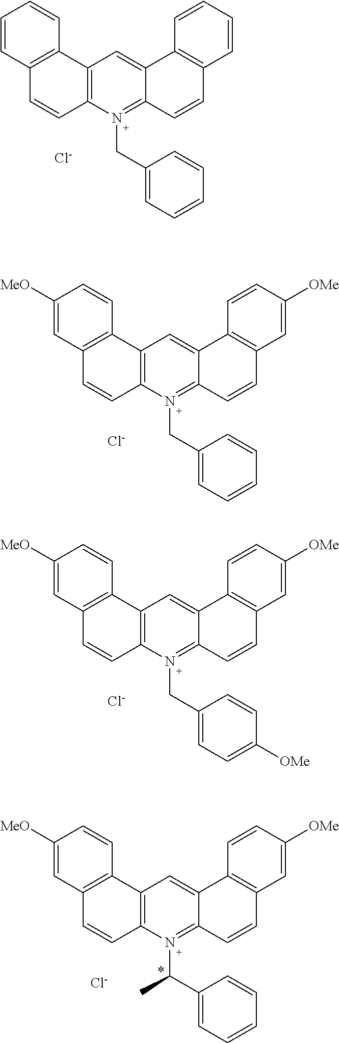 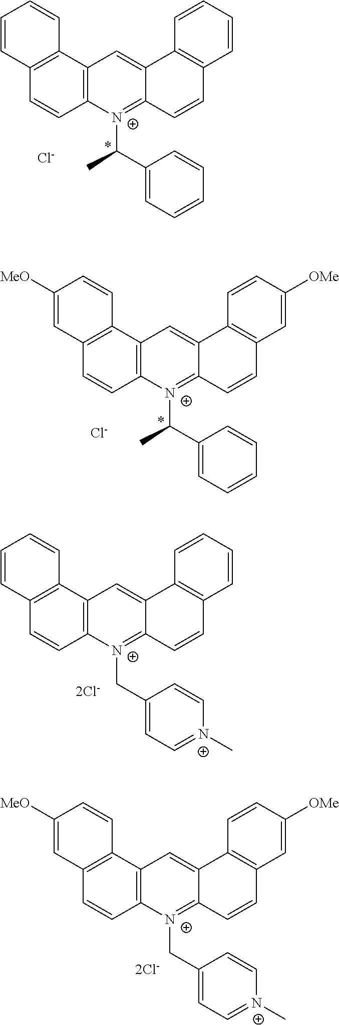
and,
wherein the compound is optionally in the form of a hydrate or a solvate.

5. A process of preparing a compound according to claim 1, comprising the step of reacting a compound of formula (II): 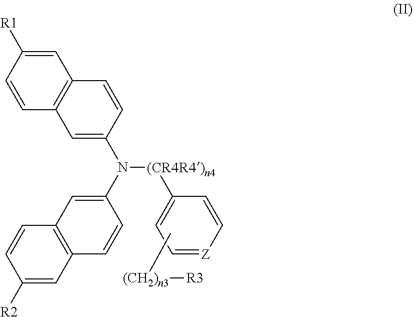
where R1, R2, R3, R4, R4′, Z, n3 and n4 are defined as in claim 1,
with a halogenating agent and DMF
and optionally isolating the compound of formula (I) that has been formed.

6. A pharmaceutical composition comprising a compound according to claim 1, and a pharmaceutically acceptable excipient.

7. A method for treating a viral infection comprising administering to a patient in need thereof a therapeutically effective amount of a compound according to claim 1.

8. The method according to claim 7, wherein the viral infection involves G-quadruplexes.

9. The method according to claim 7 wherein the viral infection is chosen from HIV, Epstein Barr virus, HPV (Papillomavirus), SARS coronavirus, Ebola virus, Marburg virus, Zika, Herpes (HHV), Hepatitis B, Hepatitis C, and Kaposi's sarcoma-associated herpesvirus (KSHV).
